(19) United States
(12) Patent Application Publication
(10) Pub. No.: US 2009/0227535 A1
(43) Pub. Date: Sep. 10, 2009

(54) Process for producing natural immunobiotic extract and uses thereof

(75) Inventors: Philip Anthony Courie, JR., Florence, KY (US); Shane Patelakis, Cornwall (CA); Amy Jo Miles, Edgewood, KY (US)

Correspondence Address: BRINKS, HOFER, GILSON & LIONE, P.O. BOX 1340, MORRISVILLE, NC, 27560, US

(73) Assignee: Progressive BioActives Inc, Charlottetown, PE (CA)

(21) Appl. No.: 11/665,461

(22) PCT Filed: Oct. 18, 2005
(86) PCT No.: PCT/CA05/01605

§ 371 Date: Dec. 10, 2007

(30) Foreign Application Priority Data
Oct. 18, 2004 (US) 10/711980

(51) Int. Cl. A61K 31/716; C07H 1/08; C12P 19/04; A61K 47/42; A61P 37/00

(52) U.S. Cl. 514/54; 536/123.12; 435/101; 514/773

(57) ABSTRACT

The present invention provides a process for producing β-(3-(1,3/1,6)-D-glucan from a cellular source comprising the steps of alkali extraction of the cellular source; water extraction; acid extraction; and water extraction, where at least one step of water extraction includes pasteurization by steam injection to a temperature of about 100° C. for 15 to about 30 minutes. The solid component produced comprises at least β-70% 1 (1,3/1,6)-D-glucan by dry weight. The present invention also provides a process of producing mannan and manno-protein complexes comprising the steps of collecting a liquid phase obtained in one an alkali extraction step of the β(3-(1,3/1,6)-D-glucan process; adjusting the pH of the liquid phase to about 5.0-8.0 with an acid; pasteurizing the liquid phase by steam injection to a temperature of about 100° C. for 15 to about 30 minutes; and isolating the mannans and manno-proteins complexes from the pasteurized liquid phase. The present invention further provides an animal feed comprising β-(3-(1,3/1,6)-D-glucan and/or mannans and manno-proteins produced according to the present process.

### FIELD OF THE INVENTION

The present, invention relates to a process of producing a natural immunobiotic extract, and uses of such extract. More specifically, the present invention is directed to a process of producing an economical and ecologically sound natural immunobiotic extract, for use as a health management instrument and a replacement for growth promotion antibiotics in livestock, poultry, companion animals and aquaculture species.

### BACKGROUND OF THE INVENTION

Antibiotic resistant bacteria have surfaced as a serious threat in the last decade due to the difficulty and expense of their eradication. The emergence of antibiotic resistant bacteria has been linked to the increased and often unwarranted use of antibiotics in humans as well as to the widespread use of antibiotics as “growth promoters” in the feed of farmed animals.

The concern with the widespread emergence of antibiotic-resistant bacteria has led the European Union to ban the use of antibiotics as “growth promoters” in animal feed. Over the last few years in the United States, a number of bills have been proposed that would ban or drastically reduce the use of antibiotics in agriculture. Because of the growing consumer awareness and concern by scientists and various governmental organizations, a ban on the use of antibiotics in agriculture may become reality in the United States and many other countries. A ban on use of growth-promoting antibiotics would certainly increase the cost of fanning animals, increase the cost of meats, and decrease meat supply unless a safe substitute for growth promotion antibiotics can be found.

For these reasons, research into the use of natural immunobiotics has gained interest. Immunobiotics are agents or organisms that promote health through broad-spectrum activation of intestinal, mucosal, or systemic immune stimulation/modulation. Enhancement of the immune system of an animal will result in a heightened ability by the animal to combat infections and diseases making the addition of antibiotic to feed unnecessary.

Among the immuno-enhancing agents that have been investigated for use in humans and animals is a β-glucan composition derived from yeast cells. Glucans, mannan and manno-proteins can be extracted from the cell walls of various yeast species, mushrooms, plants and some bacterial, lichen and algal species (reviewed in Chemistry and Biology of (1,3)-β-Glucans, B. A. Stone and A. E. Clarke, 1992, La Trobe University Press, Australia). From these sources, various different types of β-glucans can be extracted that vary in backbone composition, branching, type of monomers or substituents, resulting in polysaccharides having different physical and biological properties. For example, yeast and fingi yield a class of polysaccharides called poly-(1,3)-β-D-glucopyranosyl-(1,6)-β-D-glucopyranose, or β-(1,3/1,6) glucans, that are composed of a main chain of glucose subunits linked together in β-(1,3) glycosidic linkages and branches linked to the main chain by a β-(1,6) glycosidic linkage. The bioactivity of the β-(1,3/1,6) glucans can be related to the frequency of the β-(1,6)-branching.

β-(1,6) branched β-(1,3) glucans have been shown to activate the immune system of vertebrate as well as invertebrate organisms (Abel and Czop, “Stimulation of human monocyte beta-glucan receptors by glucan particles induces production of TNF-alpha and IL-1 Beta” (1992) Int. Journal Immunopharmacol, 14:1363-1373; Vetvicka et al, “Pilot Study: Orally-Administered Yeast β-1,3-glucan Prophylactically Protects Against Anthrax Infection and Cancer in Mice” (2002) The Journal of the American Nutraceutical Association, Vol 5, No. 2; Ueno, H., “Beta-1,3-D-Glucan,” (2000) Japanese Journal Society Terminal Systemic Diseases, 6:151-154; U.S. Pat. No. 4,138,479). β-glucan from yeast activates the immune system by binding to a specific receptor on the cell membrane of macrophages (Czop and Kay, “Isolation and Characterization of β-glucan Receptors on Human Mononuclear Phagocytes” (1991) J. Exp. Med. 173:1511-1520). The activated macrophages increase their phagocytic and bactericidal activities as well as the production of a number of cytokines, which in turn activate other components of the immune system (Di Luzio et al. in “The Macrophage in Neoplasia”, M. Fink, ed., 1976 Academic Press, New York, N.Y., pp 181-182).

Glucans that have been isolated from their natural state, demonstrate varied biological activities such as anti-infective and antibacterial (Onderdonk et al, “Anti-infective effect of poly-β-1,6 glucotriosyl-β-1,3-glucopyronose glucan in vivo” (1992) Infection and Immunity, 60:1642-1647); anti-neoplastic (Mansell et al, “Macrophage mediated destruction of human malignant cells in vivo” (1975) Journal National Cancer Institute, 54:571-80); anti-tumour (DeLuzio et al (1979) Advances in Experimental Medicine and Biology, 21A:269-290); and anti-cholesterolaemic (see for example U.S. Pat. No. 3,081,226).

Mannans and manno-protein complexes are polysaccharide complexes that are naturally occurring and may also be extracted from various yeast species, mushrooms, plants and some bacterial, lichen and algal species. Mannans are mannose polymers and represent a significant portion of the total cell wall polysaccharide component; mannans are found in covalent association with proteins, and may also comprise a phosphate component.

Mannans and manno-protein molecules are beneficial in preventing the attachment of bacteria such as Escherichia coli to the intestinal wall, thus reducing the overall infection challenge in the animal. The mannans and manno-protein complexes add additional protection and reduce overall infection challenge by preventing pathogenic organisms from attaching to the gut, thus the animal is less likely to develop an infection. Mannan has also been shown to mediate phagocytosis of material, including O-glucan, by cells of the immune system (Giaimis et al (1993) Journal of Leukocyte Biology, 54, 564-571). Thus, mannans and manno-protein complexes are also of value as immunobiotics, and may be particularly useful when combined with immuno-enhancing agents.

There have been a number of reports regarding the purification and uses of beta glucan from yeast, the process generally make use of pure Baker's or Brewer's yeast or purified cell walls and various extraction procedures involving base and acid extractions at various temperatures (see for example, Hassid et al (1941) Journal of the American Chemical Society, 63:295-298; Manners et al (1973), Biochem. J. 135:19-30). A number of methods of extracting β-(1,3/1,6)-D-glucan from yeast cells are known, including those disclosed by Jamas et al. (U.S. Pat. Nos. 4,810,646; 5,028,703; and 5,250,436), Donzis (U.S. Pat. No. 5,223,491), and Kelly (U.S. Pat. No. 6,242,594). These methods teach alkaline extraction of yeast cells, follows by acid extraction, and isolation of β-(1,3/1,6)-D-glucan. However, these methods result in β-(1,3/1,6)-D-glucan of inconsistent quality and purity, as well as varying levels of biological activity. In addition, methods are not easily adaptable to more economical large-scale batch processing, due to degradation and/or isolation of inactive forms of β-glucan. Also, prior art methods discard the mannans and manno-proteins extracted from the cell wall rather than isolating these agents, which could be beneficial for animal health.

U.S. Pat. No. 6,444,448 (Wheatcroft) discloses the preparation of insoluble yeast β-glucan-mannan complexes by autolysis. The process results in a composition comprising β-glucans, mannans and manno-proteins. However, the combination of mannan and β-glucan in this composition leads to a reduction in β-glucan bioactivity and in activation of macrophages.

Additionally, while β-(1,3/1,6)-D-glucan has shown potential as an immuno-competence enhancing agent (see for example U.S. Pat. Nos. 4,138,479; 5,817,643; 6,444,448; and 6,214,337) and a replacement for growth-promoting antibiotics, there has been less progress in establishing guidelines for supplementation, generally due to the inconsistent yield and bioactivity of prior art methods. Furthermore, economic feasibility remains an issue, given the current methods of isolating β-glucans.

### SUMMARY OF THE INVENTION

The present invention relates to a process of producing a natural immunobiotic extract, and uses of such extract. More specifically, the present invention is directed to a process of producing an economical and ecologically sound natural immunobiotic extract, for use as a health management instrument and/or a replacement for growth promotion antibiotics in livestock, poultry, companion animals and aquaculture species.

It is an object of the present invention to provide a process of producing a natural immunobiotic extract, and uses of such extract.

The present invention provides a process for producing β-(1,3/1,6)-D-glucan from a cellular source, said process comprising:
a) alkali extraction of the cellular source;
b) water extraction;
c) acid extraction; and
d) water extraction, to produce a solid component comprising at least about 70% β-(1,3/1,6)-D-glucan by dry weight.

At least one step of water extraction includes pasteurization by steam injection to a temperature of about 100° C. for a time in the range of about 15 to about 30 minutes. In the process just described, both steps of water extraction may include pasteurization.

The alkali extraction step (step a)) of the above process may comprise treating the cellular source with an alkali solution, and heating to a temperature in the range of about 45° C. to about 80° C. for about 30 minutes, followed by an increase in temperature to a temperature in the range of about 95° C. to about 150° C. for a time in the range of about 15 minutes to about 120 minutes at a pressure in the range of about 1 psi to about 25 psi. Alternatively, the alkali extraction step may comprise heating to a temperature of about 80° C. for about 45 minutes, followed by an increase in temperature to about 121° C. for about 30 minutes at a pressure in the range of about 1 psi to about 25 psi.

In the alkali extraction step of the process described above, the alkali solution may be an alkali-metal hydroxide or alkali-earth metal hydroxide solution added in a ratio in the range of about 1:5 to 1:15 cellular source to alkali solution.

The water extraction step (steps b) and d)) of the process as described above may comprise the addition of water at a ratio in the range of about 1:4 to about 1:20 solids to water, for a time in the range of about 15 minutes to about 2.5 hours at a temperature in the range of about 20° C. to about 100° C.

The acid extraction step (step c)) of the above process may comprise treating with an acid solution at a ratio in the range of about 1:4 to about 1:20 solids to acid solution, and may include heating to a temperature in the range of about 45° C. to about 120° C. for a time in the range of about 15 minutes to about 2 hours.

In the process as described above, each of steps a) to d) is followed by a step of separating the treated material into a liquid phase and a solid phase, each subsequent step being performed on the solid phase.

Optionally, the sequence of step a) followed by separation of the treated material may be performed 1, 2, or 3 times. As another option, the sequence of steps c) through d) may be performed 1, 2, or 3 times. The process as described may also include an optional step of pasteurization of the cellular source by steam injection to a temperature of about 100° C. for a time in the range of about 15 to about 30 minutes, prior to step a).

The process as described above may utilize any suitable cellular source, such as those selected from the group consisting of Baker's yeast, Brewer's yeast, spent yeast, and yeast cell wall materials.

In the above-described process, the liquid phase obtained from step a) may be collected and combined.

The present invention also provides a process for producing β-(1,3/1,6)-D-glucan from yeast, said process comprising:
a) pasteurization of the yeast by steam injection to a temperature of about 100° C. for a time in the range of about 15 to about 30 minutes;
b) separation of the pasteurized yeast into a first liquid phase and a first solid phase;
c) alkali extraction of the first solid phase with an alkali-metal or alkali-earth metal hydroxide solution in a ratio in the range of about 1:5 to about 1:15 solids to alkali solution, and heating to a temperature in the range of about 45° C. to about 80° C. for about 30 minutes;
d) increasing the temperature to a temperature in the range of about 95° C. to about 150° C. for a time in the range of about 15 min to about 120 min at a pressure in the range of about 1 psi to about 25 psi, to form an alkali-extracted mixture;
e) separation of the alkali-extracted mixture into a second liquid phase and a second solid phase;
f) water extraction of the second solid phase with water at a ratio in the range of about 1:4 to about 1:20 solids to water, for a time in the range of about 15 minutes to about 2.5 hours at a temperature in the range of about 20° C. to about 100° C., to form a water-extracted mixture;
g) pasteurization of the water-extracted mixture by steam injection to a temperature of about 100° C. for a time in the range of about 15 to about 30 minutes;
h) separation of the water-extracted mixture into a third liquid phase and a third solid phase;
i) acid extraction of the third solid phase with an acid solution in a ratio in the range of about 1:4 to about 1:20 solids to acid solution and heating to a temperature in the range of about 45° C. to about 120° C. for a time in the range of about 15 minutes to about 2 hours, to form an acid-extracted mixture;
j) separation of the acid-extracted mixture into a fourth liquid phase and a fourth solid phase;
k) water extraction of the fourth solid phase with water at a ratio in the range of about 1:4 to about 1:20 solids to water, for a time in the range of about 15 minutes to about 2.5 hours at a temperature in the range of about 20° C. to about 100° C., to form a water-extracted mixture;
l) pasteurization of the water-extracted mixture by steam injection to a temperature of about 100° C., for a time in the range of about 15 to about 30 minutes; and
m) separation of the water-extracted mixture into a fifth liquid phase and a fifth solid phase, the fifth solid phase comprising at least about 70% β-(1,3/1,6)-D-glucan by dry weight.

In the process as just described, the sequence of steps a) through e) may be performed 1, 2, or 3 times. In a further optional step, the sequence of steps i) through m) may be 1, 2, or 3 times.

The processes as described above may comprise the production of mannan and manno-protein complexes by:
i) collecting a liquid phase obtained in one or more than one alkali extraction step;
ii) adjusting the pH of the liquid phase of step i) to a pH in the range of about 5.0 to about 8.0 with an acid;
iii) pasteurizing the liquid phase of step ii) by steam injection to a temperature of about 100° C. for a time in the range of about 15 to about 30 minutes; and
iv) isolating the mannan and manno-protein complexes from the pasteurized liquid phase of step iii).

The step of isolating (step iv) in the process as just described may be accomplished by precipitation and centrifugation, or by drying.

The process for the production of mannan and manno-protein complexes as described above may yield solids in step iv) that may comprise at least about 30% mannan carbohydrate species. In addition, the solids obtained in step iv) may comprise at least about 5% protein.

The present invention further provides an animal feed comprising β-(1,3/1,6)-D-glucan produced by the process described above, in an amount effective for enhancing immuno-competence of an animal. The animal feed may be for an animal selected from the group consisting of poultry, swine, equine species such as horses, cattle, and crustaceans. The effective amount of β-glucan in the animal feed as described above may be in the range of about 5 g/1000 kg to about 500 g/1000 kg of the complete feed. The effective amount of β-(1,3/1,6)-D-glucan may vary based on the type of animal. If the animal is poultry, the effective amount may be in the range of about 20 g/1000 kg to about 50 g/1000 kg of feed. If the animal is swine, the effective amount may be in the range of about 20 g/1000 kg to about 500 g/1000 kg of feed, based on swine growth cycle and duration of use. If the animal is an equine species, the effective amount may be in the range of about 25 g/1000 kg to about 300 g/100 kg of feed. If the animal is shrimp, the effective amount may be in the range of about 35 g/1000 kg to about 300 g/1000 kg.

The present invention further provides a method of enhancing antibody formation in swine by adding an effective amount of β-(1,3/1,6)-D-glucan produced by the process as described above and feeding the animal feed to the swine.

The present invention also provides a method for enhancing the antibody formation in an animal and reducing the negative growth responses usually associated with administering a vaccine, comprising adding an effective amount of β-(1,3/1,6)-D-glucan produced by the process as described above to animal feed and feeding the animal feed to the animal.

Additionally, the present invention provides an animal feed comprising:
a) β-(1,3/1,6)-D-glucan produced according to the process of any one of claims 1 to 18 in an amount effective for enhancing immuno-competence of the animals; and
b) mannans and manno-proteins produced by process according to any one of claims 19 to 22 in an amount sufficient to inhibit bacterial adhesion to the intestinal walls of animals.

The amount of β-(1,3/1,6)-D-glucan in the animal feed as just described may be in the range of about 5 g/1000 kg to about 500 g/1000 kg of complete feed, and the amount of mannans and/or manno-proteins in the animal feed may be in the range of about 100 g/1000 kg to about 4000 g/1000 kg of complete feed.

In contrast to the processes and methods of the prior art, the present invention protects and stabilizes the β-(1,3/1,6)-D-glucan, mannan and manno-protein complexes from microbiological degradation, leading to an increased extraction efficiency and higher yields. This is turn ensures consistent quality and biological activity of the extracted polysaccharides and complexes. The recovery of the mannans and manno-protein complexes from the liquid phase recovered from the alkali extraction step in glucan extraction also lowers the cost of manufacturing. The use of the isolated β-(1,3/1,6)-D-glucan as a feed additive enhances the immune competence of farmed animals and provides an economical alternative to the current practice of antibiotic supplementation. Mannans and manno-protein complexes isolated according to the present method may be used in combination with the above β-glucan to add additional protection and reduce overall infection challenge by preventing pathogenic organisms such as Escherichia coli from attaching to the gut.

The β-(1,3/1,6)-D-glucan isolated by the method of the present invention has been shown to be capable of activating the innate immune system of animals, which allows for improved disease management. In addition, secondary health and productivity benefits, such as an increase in the number of piglets born per sow and subsequent survivability of the piglets, were observed. Treatment of animals with the β-glucan prepared in accordance with the present invention prior to administration of vaccines can boost the effectiveness of the vaccine by enhancing resulting antibody titres in animals while reducing or preventing the negative growth conditions usually attributed to the use of vaccines. It has also been shown that colostrum quality can be enhanced, resulting in enhancement of passive immunity. Thus, β-(1,3/1,6)-D-glucan lead to a reduction and/or replacement of “growth promotion” antibiotics in animal feed to maintain animals, especially farmed animals, healthy and growing at an optimal rate.

In addition, the present invention also establishes that the amount of biological activity has a direct relationship to the varying degrees of purification. Furthermore, a bell curve effect was observed in various feed trials, indicating that optimal use of β-(1,3/1,6)-D-glucan for immune modulation in livestock and other animals may not be attained by the prior art practice of using large dosages of β-(1,3/1,6)-D-glucan.

This summary of the invention does not necessarily describe all features of the invention.

### BRIEF DESCRIPTION OF TIRE DRAWINGS

These and other features of the invention will become more apparent from the following description in which reference is made to the appended drawings wherein:

FIG. 1 shows a flow chart of one embodiment of the process of the present invention.

FIG. 2 shows the structural characteristics of β-glucan as revealed by FTIR spectroscopy. FIG. 2A is the FTIR spectrum of pharmaceutical grade yeast β-glucan, and FIG. 2B is the FTIR spectrum of the β-glucan obtained by the process of the present invention.

FIG. 3 is a graph showing the comparative effects of various yeast 13-glucan compositions, including YBG (YBG Complex™, which is produced in accordance with the process of the present invention. MacroGuard™ is a commercially available product and Zymosan is a crude yeast cell wall preparation, also commercially available.

### DESCRIPTION OF PREFERRED EMBODIMENT

The present invention relates to a process for producing, a natural immunobiotic extract, and uses of such extract. More specifically, the present invention is directed to a process for producing an economical and ecologically sound natural immunobiotic extract, for use as a health management instrument and a replacement for growth promotion antibiotics in livestock and companion animals.

The following description is of a preferred embodiment.

The present invention provides a process for producing β-(1,3/1,6)-D-glucan from a cellular source, said process comprising:
a) alkali extraction of the cellular source;
b) water extraction;
c) acid extraction; and
d) water extraction, to produce a solid component comprising at least 70% β-(1,3/1,6)-D-glucan by dry weight.

In the above process, at least one step of water extraction includes pasteurization by steam injection to a temperature of about 100° C., for 15 to about 30 minutes.

By the term “β-(1,3/1,6)-D-glucan”, also referred to herein as “β-glucan”, it is meant the poly-(1,3)-β-D-glucopyranosyl-(1,6)-β-D-glucopyranose found in the cell wall of various types of cells, including, but not limited to plant, fungi and bacteria. β-glucan is composed of β-(1,3)-linked glucose units, with an inter- and intra-molecular branching via β-(1,6) linkages. β-glucan can be isolated from cellular sources containing β-(1,3/1,6)-D-glucan in the cell wall.

By “cellular source”, it is meant any appropriate source of β-(1,3)/(1,6)-D-glucan known in the art. β-glucan may be isolated from cellular sources including, but not limited to fungal, plant, and/or bacterial cells. The cellular source used as a starting material in the process of the present invention may be in any suitable form known in the art, for example, in the form of a liquid, slurry or a dry power, or may be cell wall materials derived from an appropriate fungi, plant and/or bacteria. In a non-limiting example, the cellular source is a yeast, which may be viable live or spent non-viable. The yeast or other fungal strain used may be a naturally-occurring strain, or a strain that has been genetically engineered. Any suitable yeast or fungal strain known in the art may be used, for example but without wishing to be limiting Saccharomyces spp, Shizophyllum spp, Pichia spp, Hansenula spp, Candida spp, Torulopsis spp, and Kluyveromyces spp. Specific examples of these include, but are not limited to Saccharomyces cerevisiae, Saccharomyces delbrueckii, Saccharomyces rosei, Saccharomyces microellipsodes, Saccharomyces carlsbergensis, Saccharomyces bisporus, Saccharomyces fermentati, Saccharomyces rouxii, Saccharomyces uvarum, Schizosaccharomyces pombe, Kluyveromyces polysporus, Candida albicans, Candida cloacae, Candida tropicalis, Candida utilis, Hansenula wingei, Hansenula arni, Hansenula henricii, Hansenula americana, Hansenula canadiensis, Hansenula capsulata, Hansenula polymorpha, Kluyvecomyces fragilis, Pichia kluyveri, Pichia pastoris, Pichia polymorpha, Pichia rhodanensis, Pichia ohmeri, Torulopsis bovina, and Torulopsis glabrata. Of interest as a cellular source are Saccharomyces cerevisiae, Saccharomyces delbrueckii, Saccharomyces carlsbergensis, and/or Saccharomyces rouxii, present in Baker's or Brewer's yeast, which may be viable live or spent non-viable form, and which may be obtained directly from a brewery or other suitable vendor. In a specific, non-limiting example, spent Saccharomyces cerevisiae yeast may be utilized in the process of the present invention.

The production of β-(1,3/1,6)-D-glucan from a cellular source may proceed by any suitable method of alkali extraction, water extraction, and acid extraction known in the art, the specific conditions of which may be established by a person skilled in the art. These extraction methods have been described, for example but without wishing to be limiting, by Hassid et al. (1941, Journal of the American Chemical Society, 63:295-298), Manners et al. (1973, Biochem. J. 135, 19-30), Jamas et al. (U.S. Pat. Nos. 4,810,646; 5,028,703; and 5,250,436), Donzis (U.S. Pat. No. 5,223,491), and Kelly (U.S. Pat. No. 6,242,594), all of which are incorporated herein by reference in their entirety. One non-limiting example of conditions suitable for the process of the present invention is described below.

The term “alkali extraction” (step a)), “alkaline extraction” or “alkali extracting”, refers to the treatment of the cellular source with alkali and heat to dissolve and/or extract non-β-glucan components, including mannans and manno-proteins; if cells are used as a cellular source, alkali extraction may effect cell lysis. The cellular source of β-(1,3/1,6)-D-glucan is combined with an alkaline solution, and the resulting cellular source-alkaline solution mixture may be stirred. The term “stirring” refers to any suitable method of physical agitation known in the arL For example, but without wishing to be limiting, the mixture may be stirred by a stirring apparatus, agitator, or an emulsifying pump.

The alkaline solution may be any suitable type of strong alkaline solution known in the art, for example, but not intending to be limiting, an alkali-metal hydroxide or alkali-earth metal hydroxide solution. Particular non-limiting examples of alkaline solutions are sodium hydroxide, potassium hydroxide, calcium hydroxide, and lithium hydroxide. For example, the alkaline solution may be sodium hydroxide. The alkaline solution may be of any suitable concentration, for example within the range of 0.5N to 5.0N, or any concentration therebetween, for example about 0.5N, 0.7N, 1.0N, 1.2N, 1.5N, 1.7N, 2.0N, 2.2N, 2.5N, 2.7N, 3.0N, 3.2N, 3.5N, 3.7N, 4.0N, 4.2N, 4.5N, 4.7N, and 5.0N, or a concentration in a range defined by any two concentrations disclosed herein. The alkali solution is generally added to the cellular source in a ratio in the range of about 1:3 to 1:15 cellular source to alkaline solution, or any ratio therebetween, for example about 1:3, 1:4, 1:5, 1:6, 1:7, 1:8, 1:9, 1:10, 1:11, 1:12, 1:13, 1:14, or 1:15 cellular source to alkaline solution, or a ratio in a range defined by any two ratios disclosed herein. The final pH of the cellular source-alkaline solution mixture is generally in the range of about 8 to about 14, or any pH therebetween; for example, the final pH of the cellular source-alkaline solution mixture may be about 8, 9, 10, 11, 12, 13 or 14 or a pH in a range defined by any two pH disclosed herein. In a non-limiting example, the pH of the cellular source-alkaline solution mixture is in the range of about 12 to about 14.

The cellular source-alkaline solution mixture is then heated to a temperature in the range of about 45° C. to about 120° C., or any temperature therebetween, for a time in the range of about 30 minutes to about 240 minutes, or any length of time therebetween. For example, the cellular source-alkaline solution mixture may be heated to a temperature of about 45° C., 50° C., 55° C., 60° C., 65° C., 70° C., 75° C., 80° C., 85° C., 90° C., 95° C., 100° C., 105° C., 110° C., 115° C., or 120° C., or any temperature in a range defined by the combination of any two temperatures disclosed herein, for a length of time of about 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, or 240 minutes, or any length of time in a range defined by any two times disclosed herein. For example, but without wishing to be limiting in any manner, the cellular source-alkaline solution mixture may be heated to a temperature in the range of about 45° C. to about 80° C. for a time in the range of about 30 to about 60 minutes; in a further non-limiting example, the cellular source-alkaline solution mixture may be heated to a temperature in the range of about 45° C. for about 45 minutes. During this step of heating, the cellular source-alkaline solution mixture may be stirred, as previously described.

As will be understood by a person skilled in the art, the concentration of the alkaline solution together with the temperature to which the mixture is heated will inversely affect the reaction time; for example, the higher the concentration of the alkaline solution and/or the temperature, the shorter the reaction time could be. As will also be understood by the skilled artisan, heating of the cellular source-alkaline solution mixture may result in an increase in pressure. In general and without wishing to be limiting, the pressure may increase by about 0 to about 25 psi, or any pressure therebetween; for example, the pressure may increase by about 0, 1, 2, 3, 4, 5, 6, 7, 8, 9, 10, 11, 12, 13, 14, 15, 16, 17,18, 19, 20, 21, 22, 23, 24, or 25 psi, or a pressure in a range defined by the combination of any two pressures disclosed herein.

The alkali extraction may also comprise a second step, comprising increasing the temperature of the cellular source-alkaline solution mixture, and increasing pressure. The temperature of the cellular source-alkaline solution mixture may be increased to a temperature in the range of about 95° C. to about 150° C., or any temperature therebetween, for a time in the range of about 15 to about 240 minutes, or any length of time therebetween, at a pressure in the range of about 1 psi to about 25 psi. For example, the temperature may be increased to a temperature of about 95° C., 100° C., 105° C., 110° C., 115° C., 120° C., 125° C., 130° C., 135° C., 140° C., 145° C., or 150° C., or any temperature in a range defined by the combination of any two temperatures disclosed herein, for a length of time of about 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, or 240 minutes, or any length of time in a range defined by any two times disclosed herein, at a pressure in of about 1, 2, 3, 4, 5, 6, 7, 8, 9, 10, 11, 12, 13, 14, 15, 16, 17, 18, 19, 20, 21, 22, 23, 24, or 25 psi, or any pressure in a range defined by any two pressures disclosed herein. For example, and without wishing to be limiting in any manner, the temperature may be increased to a temperature in the range of about 95° C. to about 150° C., for a time in the range of about 15 to about 120 minutes, at a pressure in the range of about 1 psi to about 25 psi; in another non-limiting example, the temperature may be increased to about 121° C., for about 30 minutes, at a pressure in the range of about 1 psi to about 15 psi. During this step of heating, the cellular source-alkaline solution mixture may be stirred, as described above.

Again, a person skilled in the art will understand that the temperature to which the mixture is heated will inversely affect the reaction time; for example, the higher the concentration of the alkaline solution and/or the temperature, the shorter the reaction time could be.

The method of alkali extraction as described above results in an alkali-extracted mixture. The alkali-extracted mixture or the pooled alkali-extracted mixtures are then separated.

By the term “separated” or “separation”, it is meant that the mixture in question is divided into its liquid and solid components. The liquid and solid components may also be referred to herein as “liquid phase” and “solid phase”. Any suitable method of separation known in the art may be used. For example, but without wishing to be limiting in any manner, the solid and liquid components may be separated by centrifugation, filtration, membrane filtration, or reverse osmosis. In a particular, non-limiting example, the mixture may be separated by centrifugation.

The liquid phase obtained after separation of the alkali-extracted mixture, the “alkali-extracted liquid phase”, contains most alkaline-soluble non-targeted β-glucan components and non-β-glucan components of the cellular source. The alkali-extracted liquid phase is collected and pooled, and may be further processed to obtain mannans and manno-proteins, as described below. The solid phase obtained after alkali extraction, the “alkali-extracted solid phase”, contains β-glucan.

A person skilled in the art will recognize that, optionally, repeated rounds of alkali extraction may be performed on “fresh” cellular source material. By “fresh” material, it is meant cellular source that has not previously been submitted to alkaline extraction. For example, and without wishing to be limiting in any manner, alkali extraction may be performed 1 to 20 times, or any amount of repetitions therebetween; for example, the alkali extraction may be repeated 1, 2, 3, 4, 5, 6, 7, 8, 9, 10, 11, 12, 13, 14, 15, 16, 17, 18, 19, or 20 times, or any amount of repetitions defined by a range of any two numbers disclosed herein. Without wishing to be limiting, the alkali extraction step may be performed, for example, 1, 2 or 3 times. In the event that the alkali extraction step is performed on fresh cellular source material, the alkali-extracted solid phases from each round of alkaline extraction are pooled.

A person skilled in the art will also recognize that successive rounds of alkali extraction may optionally be performed, as required, to increase the removal of non-targeted β-glucan components and non-β-glucan components. In this case, alkali extraction is performed on the alkali-extracted solid phase or the pooled alkali-extracted solid phase. For example, and without wishing to be limiting in any manner, alkali extraction may be successively performed 1 to 20 times, or any amount of repetitions therebetween, as required; for example, the alkali extraction may be performed 1, 2, 3, 4, 5, 6, 7, 8, 9, 10, 11, 12, 13, 14, 15, 16, 17, 18, 19, or 20 times, or any amount of repetitions defined by a range of any two numbers disclosed herein. Without wishing to be limiting, the alkali extraction step may be performed, for example, 1, 2 or 3 times. As would be understood by a skilled person, the successive rounds of alkali extraction will increase the purity of the β-glucan in the alkali-extracted solid phase; however, the overall cost of the process will increase with each successive round of alkali extraction. Therefore, a person skilled in the art must consider the balance between the number of alkaline extractions and the economic viability of the process.

The alkali-extracted solid phase or the pooled alkali-extracted solid phase is then submitted to water extraction (step b)). The term “water extraction”, which is also known in the art as “water wash”, refers to the washing of the solid component with water to remove any residual non-β-glucan components; the water extraction step also serves to lower the pH of the alkali-extracted solid phase. The water extraction step may be performed by any suitable method known in the art. For example, and without wishing to be limiting in any manner, the solid component may be resuspended in water at a ratio in the range of about 1:4 to about 1:20 solid component to water, or any ratio therebetween; for example, water may be added to the solid phase at a ratio of about 1:4, 1:5, 1:6, 1:7, 1:8, 1:9, 1:10, 1:11, 1:12, 1:13, 1:14, 1:15, 1:16, 1:17, 1:18, 1:19, or 1:20 solid component to water, or a ratio in a range defined by any two ratios disclosed herein. The resuspended solids are heated to a temperature in the range of about 20° C. to about 100° C., or any temperature therebetween, for a time in the range of about 15 minutes to about 240 minutes, or any amount of time therebetween. For example, the resuspended solids may be heated at a temperature of about 20° C., 25° C., 30° C., 35° C., 40° C., 45° C., 50° C., 55° C., 60° C., 65° C., 70° C., 75° C., 80° C., 85° C., 90° C., 95° C., 100° C., or any temperature in a range defined by the combination of any two temperatures disclosed herein, for a length of time of about 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, or 240 minutes, or any length of time in a range defined by any two times disclosed herein. For example, but without wishing to be limiting, the resuspended solids may be heated to a temperature in the range of about 20° C. to about 100° C., for a time in the range of about 15 to about 150 minutes; in a further non-limiting example, resuspended solids may be heated to a temperature of about 20° C. to about 60° C. for about 30 minutes.

As will be understood by a person skilled in the art the temperature to which the mixture is heated will inversely affect the reaction time; for example, the higher the concentration of the alkaline solution and/or the temperature, the shorter the reaction time could be. During water extraction, the resuspended solids may be stirred by any suitable method known in the art, as previously described. Water extraction produces a water-extracted mixture.

The water-extracted mixture is then separated into a liquid phase and a solid phase, in a manner as previously described. The liquid phase of the water-extracted mixture is generally discarded, and the solid phase is retained for acid extraction.

As would be evident to a person skilled in the art, successive rounds of water extraction may optionally be performed, as required, until all yeast solids have been separated. In this case, water extraction is performed on the water-extracted solid phase. For example, and without wishing to be limiting in any manner, water extraction may be successively performed 1 to 10 times, or any amount of repetitions therebetween, as required; for example, the water extraction may be performed 1, 2, 3, 4, 5, 6, 7, 8, 9, 10, times, or any amount of repetitions defined by a range of any two numbers disclosed herein. Without wishing to be limiting, the alkali extraction step may be performed, for example, 1, 2 or 3 times. However there is a balance between number of water washes and the economic viability of process. The solid phase resulting from the successive water extraction steps is then submitted to acid extraction (step c)).

The term “acid extraction”, “acidic extraction”, or “acid extracting”, refers to the treatment of the solid phase of the water-extracted mixture with an acid and heat to dissolve and/or extract any residual non-targeted β-glucan components and non-β-glucan components, including but not limited to other polysaccharides/sugars and some lipids. The solid phase of the water-extracted mixture is combined with an acid solution to form a solid phase-acidic solution mixture, and may be stirred. Stirring may be accomplished by any suitable method known in the art, as described above.

The acid solution may be any suitable type of acid solution known in the art, for example, but not intending to be limiting, any mild acid solution. Of interest for use in acid extraction is acetic acid. The acid solution may be of any suitable concentration, for example within the range of 2% to 10% (v/v), or any concentration therebetween; for example, the acid solution may be a 2%, 2.5%, 3%, 3.5%, 4%, 4.5%, 5%, 5.5%, 6%, 6.5%, 7%, 7.5%, 8%, 8.5%, 9%, 9.5%, or 10% (v/v) acid solution, or a concentration in a range defined by any two concentrations disclosed herein. In a non-limiting example, the acid solution is a 3% solution. The acid solution is generally added to the solid phase of the water-extracted mixture in a ratio in the range of about 1:4 to about 1:20 solid component to acid solution, or any ratio therebetween; for example, the acid solution may be added to the solid phase at a ratio of about 1:4, 1:5, 1:6, 1:7, 1:8, 1:9, 1:10, 1:11, 1:12, 1:13, 1:14, 1:15, 1:16, 1:17, 1:18, 1:19, or 1:20 solid component to acid solution, or a ratio in a range defined by any two ratios disclosed herein. In a non-limiting example, acid solution is added in a ratio of 1:10 solid component to acid solution. The final pH of the solid phase-acid solution mixture is generally in the range of about 2 to about 5, or any pH therebetween; for example, the final pH of the cellular source-alkaline solution mixture may be about 2, 3, 4, or 5, or a pH in a range defined by any two pH disclosed herein. In a non-limiting example, the pH of the solid phase-acid solution mixture is in the range of about 3 to about 4, or in a further example, is about 4.

The solid phase-acid solution mixture is then heated to a temperature in the range of about 45° C. to about 120° C., or any temperature therebetween, for a time in the range of about 15 minutes to about 120 minutes, or any length of time therebetween. For example, the solid phase-acid solution mixture may be heated to a temperature of about 45° C., 50° C., 55° C., 60° C., 65° C., 70° C., 75° C., 80° C., 85° C., 90° C., 95° C., 100° C., 105° C., 110° C., 115° C., or 120° C., or any temperature in a range defined by the combination of any two temperatures disclosed herein, for a length of time of about 15, 20, 25, 30, 35, 40, 45, 50, 55, 60, 65, 70, 75, 80, 85, 90, 95, 100, 105, 110, 115, or 120 minutes, or any length of time in a range defined by any two times disclosed herein. For example, but without wishing to be limiting in any manner, the solid phase-acid solution mixture may be heated to a temperature in the range of about 45° C. to about 80° C. for a time in the range of about 15 to about 60 minutes; in a further non-limiting example, the solid phase-acid solution mixture may be heated to a temperature of about 80° C. for about 60 minutes.

As will be understood by a person skilled in the art, the concentration of the acid solution together with the temperature to which the mixture is heated will inversely affect the reaction time; for example, the higher the concentration of the acid solution and/or the temperature, the shorter the reaction time could be. As will also be understood by the skilled artisan, heating of the solid phase-acid solution mixture may result in an increase in pressure. In general and without wishing to be limiting, the pressure may increase by about 0 to about 25 psi, or any pressure therebetween; for example, the pressure may increase by about 0, 1, 2, 3, 4, 5, 6, 7, 8, 9, 10, 11, 12, 13, 14, 15, 16, 17,18, 19, 20, 21, 22, 23, 24, or 25 psi, or a pressure in a range defined by the combination of any two pressures disclosed herein.

The method of acid extraction as described above results in an acid-extracted mixture. The acid-extracted mixture is then separated, as previously described. The liquid phase obtained after separation of the acid-extracted mixture is discarded. The solid phase obtained after acid extraction, the “acid-extracted solid phase”, contains β-glucan.

A person skilled in the art will recognize that repeated rounds of acid extraction may optionally be performed on water-extracted solid phase that has not previously been submitted to acid extraction. For example, and without wishing to be limiting in any manner, acid extraction may be performed 1 to 20 times, or any amount of repetitions therebetween; for example, the acid extraction may be performed 1, 2, 3, 4, 5, 6, 7, 8, 9, 10, 11, 12, 13, 14, 15, 16, 17, 18, 19, or 20 times, or any amount of repetitions defined by a range of any two numbers disclosed herein. Without wishing to be limiting, the acid extraction step may be performed, for example, 1, 2 or 3 times. In the event that the acid extraction step is performed on water-extracted solid phase that has not previously been submitted to acid extraction, the acid-extracted solid phases from each round of acidic extraction are pooled.

A person skilled in the art will also recognize that successive rounds of acid extraction may optionally be performed, as required, to remove non-β-glucan components. In this case, acid extraction is performed on the acid-extracted solid phase or the pooled acid-extracted solid phase. For example, and without wishing to be limiting in any manner, acid extraction may be successively performed 1 to 20 times, or any amount of repetitions therebetween, as required; for example, the acid extraction may be performed 1, 2, 3, 4, 5, 6, 7, 8, 9, 10, 11, 12, 13, 14, 15, 16, 17, 18, 19, or 20 times, or any amount of repetitions defined by a range of any two numbers disclosed herein. Without wishing to be limiting, the acid extraction step may be performed, for example, 1, 2 or 3 times. As would be understood by a skilled person, the successive rounds of acid extraction will increase the purity of the β-glucan in the acid-extracted solid phase; however, the overall cost of the process will increase with each successive round of acid extraction. Therefore, the person skilled in the art must consider the balance between the number of alkaline extractions and the economic viability of the process.

The acid-extracted solid phase or the pooled acid-extracted solid phase, is then submitted to water extraction (step d)). The water extraction of the acid-extracted solid phase or the pooled acid-extracted solid phase may proceed under conditions as previously described, resulting in a water-extracted mixture. The water-extracted mixture is then separated into a liquid phase and a solid phase, by a method as described above. The liquid phase of the water-extracted mixture is generally discarded, and the solid phase is retained. As previously described, and as would be evident to a person skilled in the art, water extraction may optionally be repeated, as required, until all yeast solids have been separated. In the case of repeatedly performed water extractions, the solid phases resulting from the water extractions are pooled.

In the method of the present invention, whether by the conditions for alkali extraction, acid extraction and water extraction described above, or by prior art conditions, at least one water extraction step includes a step of pasteurization prior to separation. For example, the water extraction step (step b)) following the alkali extraction step (step a)) may include pasteurization, the water extraction step (step d)) following the acid extraction step (step c)) may include pasteurization, or both the water extraction step (step b)) following the alkali extraction step and the water extraction step (step d)) following the acid extraction step may include pasteurization.

By the term “pasteurization” or “pasteurize”, it is meant the treatment of the solids resuspended in water to stabilize the mixture, and minimize microbial degradation of the β-(1,3/1,6)-D-glucan. Pasteurization may be done by any method known in the art, for example, but not limited to, direct steam injection or indirect steam injection, for example using a steam jacket. For example, but without wishing to be limiting, pasteurization of the water-extracted mixture may occur to a temperature of about 75° C. to about 100° C., or any temperature therebetween, for about 15 to about 240 minutes, or any length of time therebetween. For example, pasteurization of the water-extracted mixture may occur to a temperature of about 75° C., 78° C., 80° C., 82° C., 85° C., 88° C., 90° C., 92° C., 95° C., 98° C., or 100° C., or any temperature in a range defined by the combination of any two temperatures disclosed herein, for a time of about 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, or 240 minutes, or any length of time in a range defined by any two times disclosed herein. Without wishing to be limiting, pasteurization may occur to a temperature of about 85° C. to about 100° C. for about 15 to about 30 minutes; in a further non-limiting example, pasteurization may occur to a temperature of about 100° C. for about 20 minutes.

As will be understood by a person skilled in the art, the temperature to which the mixture is pasteurized will inversely affect the reaction time; for example, the higher the temperature, the shorter the reaction time could be.

Once the water-extracted mixture has been pasteurized, separation of the mixture into liquid and solid phases can proceed, as previously described.

Optionally, the pasteurized water-extracted mixture following either the alkali extraction step, the acid extraction step, or both the alkali and acid extractions steps may be stirred by any suitable method known in the art, as previously described, for about 2 hours to about 7 days, or any amount of time therebetween, prior to separation. For example, the pasteurized water-extracted mixture may be stirred for about 2 hours, 4 hours, 6 hours, 8 hours, 10 hours, 12 hours, 14 hours, 16 hours, 18 hours, 20 hours, 22 hours, 1 day, 1.5 days, 2 days, 2.5 days, 3 days, 3.5 days, 4 days, 4.5 days, 5 days, 5.5 days, 6 days, 6.5 days or 7 days, or an amount of time defined by a range of any two amounts disclosed herein. In a non-limiting example, the water-extracted mixture may be stirred for about 2 hours to 2 days at ambient temperature. Stirring of the pasteurized water-extracted mixture prior to separation allows the accumulation of pasteurized water-extracted mixture from separate processes, such that the final separation step may proceed on a larger scale. As the water-extracted mixture is pasteurized, degradation of the β-glucan components is prevented or minimized.

In another optional step, the process of the invention as described above may also comprise a pre-treatment step. For example, the cellular source may be pre-treated by pasteurization prior to the alkali extraction step (step a)). In this case, the cellular source may be provided as a yeast slurry, cream, packed yeast cake. The slurry, cream, or yeast cake may comprise a solid content in the range of about 15% to 80% solids, or any amount therebetween; for example, the slurry may comprise 15%, 20%, 25%, 30%, 35%, 40%, 45%, 50%, 55%, 60%, 65%, 70%, 75% or 80% solids, or any percentage of solids in a range defined by the combination of any two percentages disclosed. In a non-limiting example, the slurry may comprise a solids content in the range of about 60% to about 70% solids. Pasteurization in the pre-treatment step is performed generally as previously described, and may optionally be followed by a water extraction step.

Separation of the water-extracted mixture obtained in step d) of the above process results in a solid component comprising a percentage of β-(1,3/1,6)-D-glucan in the range of least about 70% to about 98% by dry weight, or any percentage therebetween; for example, the solid component may comprise about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, or 98% β-(1,3/1,6)-D-glucan by dry weight, or any percentage in a range defined by the combination of any two percentages disclosed herein. In a non-limiting example, the solid component may comprises about 70 to about 90% β-(1,3/1,6)-D-glucan by dry weight, or in a further example, may comprise 80% β-(1,3/1,6)-D-glucan by dry weight.

The final β-(1,3/1,6)-D-glucan composition prepared according to the present process has a biological activity of at least about 30 μg Bb released per mg of β-(1,3/1,6)-D-glucan, or any activity therebetween, as determined by the alternative complement activation experiment (National Jewish Medical & Research Center, Denver, Colo.). For example, the β-(1,3/1,6)-D-glucan composition may have an activity of at least about 30, 31, 32, 33, 34, 35, 36, 37, 38, 39, 40, 41, 42, 43, 44, 45, 46, 47, 48, 49, or 50 μg Bb released per mg of β-(1,3/1,6)-D-glucan, or an activity in a range defined by any two activities disclosed herein. In a particular, non-limiting example, the final β-(1,3/1,6)-D-glucan composition has an activity of at least 40 μg Bb released per mg of β-(1,3/1,6)-D-glucan.

After the final separation step, the solid component may be dried by any suitable method known in the art. The term “drying” refers to the removal of water (moisture) or solvent. Drying of the solid component yields the final β-glucan product, and may be performed by any suitable method known in the art. For example, and without wishing to be limiting in any manner, the solid component may be dried by lyophilization, heating, air-drying, drum-drying, spray-drying, IR drying, drying by microwave or radiowave, drying by radiant heat, or any other suitable method. In a non-limiting example, the solid component may be dried by spray-drying.

The final solid component may be dried to a moisture content of less than about 10%, or any percentage therebetween; for example the moisture content of the final product may be less than about 3%, 4%, 5%, 6%, 7%, 8%, 9%, or 10%, or any moisture content in a range defined by the combination of any two percentages disclosed herein. In a specific, non-limiting example, the moisture content of the final product has a moisture content of less than about 10%.

The dried final product, a β-(1,3/1,6)-D-glucan composition, is a powder comprising particles with an average diameter of less than about 7 μm; for example, the average particle size may be less than about 7 μm, 6.5 μm, 6 μm, 5.5 μm, 5 μm, 4.5 μm, 4 μm, 3.5 μm, 3 μm, 2.5 μm, 2 μm, 1.5 μm, or 1 μm, or any size in a range defined by the combination of any two sizes disclosed herein. The powder may be further processed to obtain particles of a desired size. For example, but without wishing to be limiting, the powder may be milled, by hammer milling or ball milling.

The dried final β-(1,3/1,6)-D-glucan composition is stable, and may have a shelf-life of at least about 12 months when stored at a temperature in the range of about 15° C. to about 25° C. in sealed container. For example, the shelf-life of the β-glucan of the present invention may be of at least about 12, 13, 14, 15, 16, 17, 18, 19, 20, 21, 22, 23, 24, 25, 26, 27, 28, 29, 30, 31, 32, 33, 34, 35, or 36 months, or any shelf-life in a range defined by the combination of any two times disclosed herein, when stored at a temperature of about 15° C., 16° C., 17° C., 18° C., 19° C., 20° C., 21° C., 22° C., 23° C., 24° C., or 25° C., or any temperature in a range defined by the combination of any two temperatures disclosed herein. In a non-limiting example, the final β-glucan composition has a shelf-life of at least about 24 months when stored at a temperature in the range of about 20 to about 25° C. in sealed container. The sealed container may be a any suitable container known in the art, for example, but without wishing to be limiting in any manner, may be a container or a bag made of any suitable material, for example plastic, that will prevent contact with humidity.

The present invention also provides a process for producing mannan and manno-protein complexes from a cellular source, comprising:
i) collecting the liquid phase obtained from one, or more than one alkali extraction step (step a)) of the process for producing β-(1,3/1,6)-D-glucan described above;
ii) adjusting the pH of the liquid phase of step i) to about 5.0-8.0 with an acid;
iii) pasteurizing the liquid phase of step ii) by steam injection to a temperature of about 100° C. for 15 to about 30 minutes; and
iv) isolating the mannan and manno-protein complexes from the pasteurized liquid phase.

By the term “mannans”, it is meant the class of polysaccharides represented by mannose polymers; mannans are found primarily in covalent association with proteins, in complexes called “manno-protein complexes”, also referred to herein as “manno-proteins”. These types of polysaccharide complexes are found in the cell wall of various types of cells, including, but not limited to plant, yeast, fungi and bacteria, and may be isolated from any such suitable cellular source known in the art. In a non-limiting example, the cellular source fungi (for example, yeast), and may be a naturally-occurring strain, or a strain that has been genetically engineered. Any suitable yeast or fungal strain known in the art may be used, for example but without wishing to be limiting Saccharomyces spp, Shizophyllum spp, Pichia spp, Hansenula spp, Candida spp, Torulopsis spp, and Kluyveromyces spp. Specific examples of these include, but are not limited to Saccharomyces cerevisiae, Saccharomyces delbrueckii, Saccharomyces rosei, Saccharomyces microellipsodes, Saccharomyces carisbergensis, Saccharomyces bisporus, Saccharomyces fermentati, Saccharomyces rouxii, Saccharomyces uvarum, Schizosaccharomyces pombe, Kluyveromyces polysponis, Candida albicans, Candida cloacae, Candida tropicalis, Candida utilis, Hansenula wingei, Hansenula arni, Hansenula henricii, Hansenula americana, Hansenula canadiensis, Hansenula capsulata, Hansenula polymorpha, Kluyvecomyces fragils, Pichia kluyveri, Pichia pastoris, Pichia polymorpha, Pichia rhodanensis, Pichia ohmeri, Torulopsis bovina, and Torulopsis glabrata. Of interest as a cellular source are Saccharomyces cerevisiae, Saccharomyces delbrueckhi, Saccharomyces carlsbergensis, and/or Saccharomyces rouxii, present in Baker's or Brewer's yeast, which may be viable live or spent non-viable form, and which may be obtained directly from a brewery or other suitable vendor. In a specific, non-limiting example, Saccharomyces cerevisiae yeast may be utilized in the process of the present invention.

The mannan and manno-protein complexes in the process of the present process are isolated from the liquid phase obtained from one, or more than one, alkali extraction step (step a)) of the process for producing β-(1,3/1,6)-D-glucan previously described.

As previously described, the alkali-extracted liquid phase contains most alkaline-soluble non-β-glucan components of the cellular source, including mannans and manno-proteins. The alkali-extracted liquid phase obtained from one, or more than one, alkali extraction step is collected and may be pooled, as required.

The pH of the one, or more than one, alkali-extracted liquid phase is then adjusted to a pH in the range of about 5.0 to about 8.0, or any pH therebetween, using an acid. For example, the pH of the alkali-extracted liquid phase may be adjusted to about 5.0, 5.2, 5.5, 5.7, 6.0, 6.2, 6.5, 6.7, 7.0, 7.2, 7.5, 7.7, or 8.0, or any pH in a range defined by any two pH disclosed herein. For example, and without wishing to be limiting, the pH of the alkali-extracted liquid phase may be adjusted to about 7.0. Any suitable acid known in the art may be used to adjust the pH, for example, but without wishing to be limiting in any manner, any strong acid known in the art may be used. For example, hydrochloric acid, nitric acid, or sulfuric acid may be used to adjust the pH of the liquid. In a further, non-limiting example, hydrochloric acid (HCl) may be used to adjust the pH.

The alkali-extracted liquid phase may be stirred during, after or both during and after adjustment of the pH. The term “stirring” refers to any suitable method of physical agitation known in the art. For example, but without wishing to be limiting, the mixture may be stirred by a stirring apparatus, agitator, or an emulsifying pump.

The pH-adjusted, alkali-extracted liquid phase is then pasteurized. The pasteurization step is performed in a manner as previously described. For example, and without wishing to be limited in any manner, pasteurization may be accomplished by any method known in the art, for example, but not limited to, direct steam injection or indirect steam injection, for example using a steam jacket. For example but without wishing to be limiting, pasteurization of the water-extracted mixture may occur to a temperature of about 75° C. to about 100° C., or any temperature therebetween, for about 15 to about 240 minutes, or any length of time therebetween. For example, pasteurization of the water-extracted mixture may occur to a temperature of about 75° C., 78° C., 80° C., 82° C., 85° C., 88° C., 90° C., 92° C., 95° C., 98° C., or 100° C., or any temperature in a range defined by the combination of any two temperatures disclosed herein, for a time of about 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, or 240 minutes, or any length of time in a range defined by any two times disclosed herein. Without wishing to be limiting, pasteurization may occur to a temperature of about 85° C. to about 100° C. for about 15 to about 30 minutes; in a further non-limiting example, pasteurization may occur to a temperature of about 100° C. for about 20 minutes.

As will be understood by a person skilled in the art, the temperature to which the mixture is pasteurized will inversely affect the reaction time; for example, the higher the temperature, the shorter the reaction time will be.

Following pasteurization, mannans and manno-proteins complexes are isolated from the pasteurized, pH-adjusted, alkali-extracted liquid phase. Isolation of the molecules may be accomplished by any suitable method known in the art, for example by precipitation or by drying.

Drying of the pH-adjusted, alkali-extracted liquid phase may be performed by any suitable method known in the art. For example, and without wishing to be limiting in any manner, the solid component may be dried by lyophilization, heating, air-drying, drum-drying, spray-drying, IR drying, drying microwave or radiowave, drying by radiant heat, or any other suitable method. In a non-limiting example, the solid component may be dried by spray-drying. Drying of the liquid phase yields a mannan and manno-protein product.

Alternatively, the mannan and manno-proteins may be isolated by precipitation of the liquid phase; precipitation of the liquid phase may be accomplished by any suitable method know in the art, for example using alcohol. Any suitable food grade alcohol may be employed, for example, but not limited to ethanol or propanol. In accordance with methods known in the art, the amount of alcohol used may be in the range of about 1:0.25 to about 1:3 liquid to alcohol. The precipitated mannan and manno-proteins are centrifuged and the liquid phase is discarded; the mannan and manno-protein precipitate may then be dried by any suitable method known in the art to yield the mannan and manno-protein product.

Isolation of the mannan and manno-proteins in step iv) of the above process results in a final product comprising a percentage of mannan carbohydrate species in the range of least about 25% by dry weight; for example, the final product may comprise at least about 25%, 26%, 27%, 28%, 29%, 30%, 31%, 32%, 33%, 34%, 35%, 36%, 37%, 38%, 39%, or 40% mannan carbohydrate species by dry weight, or any percentage in a range defined by the combination of any two percentages disclosed herein. In a non-limiting example, the final mannan and manno-protein product comprises at least about 30% mannan carbohydrate species by dry weight. In addition, the final product may comprise at least about 5% protein by dry weight; for example, the solid component may comprise at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30% by dry weight. In a non-limiting example, the final mannan and manno-protein product comprises at least 5% protein by dry weight. Thus, the final product may comprise of at least about 35% manno-proteins by dry weight; for example, the final product may comprise at least about 30%, 31%, 32%, 33%, 34%, 35%, 36%, 37%, 38%, 39%, 40%, 41%, 42%, 43%, 44%, 45%, 46%, 47%, 48%, 49% or 50% manno-proteins by dry weight.

The mannan and manno-protein product may be dried to a moisture content of less than about 15%, or any percentage therebetween; for example the moisture content of the final product may be less than about 3%, 4%, 5%, 6%, 7%, 8%, 9%, 10%, 11%, 12%, 13%, 14%, or 15%, or any moisture content in a range defined by the combination of any two percentages disclosed herein. In a specific, non-limiting example, the moisture content of the final product has a moisture content of less than about 15%.

The dried mannan and manno-protein product is a powder and may be further processed to obtain a desired particle size. For example, but without wishing to be limiting, the powder may be milled, by hammer milling or ball milling.

The present invention also pertains to an animal feed comprising β-(1,3/1,6)-D-glucan produced by the process as described above. The β-(1,3/1,6)-D-glucan may be; added to the animal feed in an amount effective for enhancing the immuno-competence of the animal in question. The term “enhance immuno-competence” refers to enhancing the innate immune system of animals in a non-specific manner. β-(1,3/1,6)-D-glucan activates the immune system by binding to specific receptors on the cell membrane of macrophages and other immune cells, which then increase their phagocytic and bactericidal activities and/or the production of a number of cytokines, which in turn activate other components of the immune system.

As will be understood by a person skilled in the art, the effective amount of β-glucan will vary based on the type of animal. The animal feed of the present invention may be destined for any type of livestock, poultry, fish, crustaceans, shrimp or companion animals. For example, but without wishing to be limiting in any manner, the animal feed may be used for feeding avian species such as poultry, swine, equine species such as horses, cattle, goats, sheep, and other livestock, companion animals including fish, dogs, cats and aquaculture species such as crustaceans, shrimp and farmed fish. In general, the effective amount of β-(1,3/1,6)-D-glucan will be in the range of about 5 g/1000 kg of complete feed to about 500 g/1000 kg of complete feed, or any amount therebetween. For example, the effective amount of β-(1,3/1,6)-D-glucan may be 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 g/1000 kg of complete feed, or any amount in a range defined by any two amounts disclosed herein. In more specific examples, without wishing to be limiting in any manner, the following effective amounts of β-glucan may be used:
where the animal is poultry, the effective amount may be between about 20 and about 50 g/1000 kg of complete feed, for example about 40 g/1000 kg of complete feed;
where the animal is swine, the effective amount may be between about 20 to about 500 g/1000 kg of complete feed, based on swine growth cycle; for example between about 75 to about 95 g/1000 kg of complete feed, or 80 g/1000 kg of complete feed, for a lactating swine and piglets; or in a further example, the effective amount may be about 150 to about 450 g/1000 kg of complete feed for gestating swine, or about 200 to about 400 g/1000 kg of complete feed, depending on duration and gestation period; in a non-limiting example, a gestating swine may be fed about 200 g/1000 kg complete feed throughout gestation, or may be fed about 400 g/000 kg complete feed during the last about 30 to about 40 days of gestation;
where the animal is an equine species such as a horse, the effective amount may be between about 25 to about 300 g/1000 kg of complete feed, for example, between about 25 to about 100 g/1000 kg of complete feed, or in a further example, the effective amount may be about 60 g/1000 kg of complete feed;
where the animal is shrimp, the effective amount may be between about 35 to about 300 g/1000 kg of complete feed, for example about 100 g/1000 kg of complete feed.

The present invention further provides an animal feed comprising: a) β-(1,3/1,6)-D-glucan produced according to the process described above, in an amount effective for enhancing immuno-competence of animal; and b) mannans and manno-proteins produced by the process described above in an amount sufficient to reduce or inhibit bacterial adhesion to the intestinal walls of animals. For example, the animal feed may comprise an amount of β-(1,3/1,6)-D-glucan in the range of about 5 to about 500 g/1000 kg of complete feed, or any amount therebetween, and an amount of mannans and/or manno-proteins in the range of about 100 to about 4000 g/1000 kg of complete feed, or any amount therebetween; for example, the animal feed may comprise β-(1,3/1,6)-D-glucan in an amount of about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495 or 500 g/1000 kg of complete feed, or any amount in a range defined by any two amounts disclosed herein, and mannans and/or manno-proteins in an amount of about 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, 3600, 3650, 3700, 3750, 3800, 3850, 3900, 3950, or 4000 g/1000 kg of complete feed, or any amount in a range defined by any two amounts disclosed herein.

In field trials using the β-(1,3/1,6)-D-glucan produced by the process of the present invention, a dosage-dependent response or bell-curve effect was observed in various feed trials, particularly in swine. Specifically, piglets vaccinated using a commercial PRRS vaccine and subsequently fed β-glucan in dosages of either 0, 40, 80 or 120 g/1000 kg of complete feed showed a dosage-dependent response, where 80 g/1000 kg of complete feed maximized the antibody response and average daily gain, while 120 g/1000 kg of complete fee gave a response similar to the control (see Example 4).

In another trial, sows were fed either 0, 0.5, or 1 g β-glucan/sow/day for 4 weeks prior to farrowing and were vaccinated with a commercial oil-adjuvant Mycoplasma hyopneumoniae 14 days prior to farrowing. Sows fed β-glucan at 1 g/day showed a significant increase in the passive transfer of anti-Mycoplasma antibodies to piglets. A dosage of 0.5 g β-glucan/sow/day showed an antibody response that was not significantly different from that of the control (see Example 6).

Without wishing to be bound by theory, the mechanism through which the bell curve effect is obtained may be related to a biological feed-back mechanism that down regulates immune function at high dosages. This is an important discovery and direct commercial implications for the proper and optimal use of purified β-glucan for immune modulation in livestock/animals. This is also in contradiction with the prior art practice of erroneously recommending large dosages in the range of 1 to 2 kg/1000 kg complete feed, which may be ineffective and/or produce inconsistent results. Thus, the extraction process used, the purity and the dosage of β-glucan appear to be factors in its optimal application.

The invention will now be described in detail by way of reference only to the following non-limiting examples.

### Example 1

### Purification of β-(1,3/1,6)-D-Glucan from Yeast

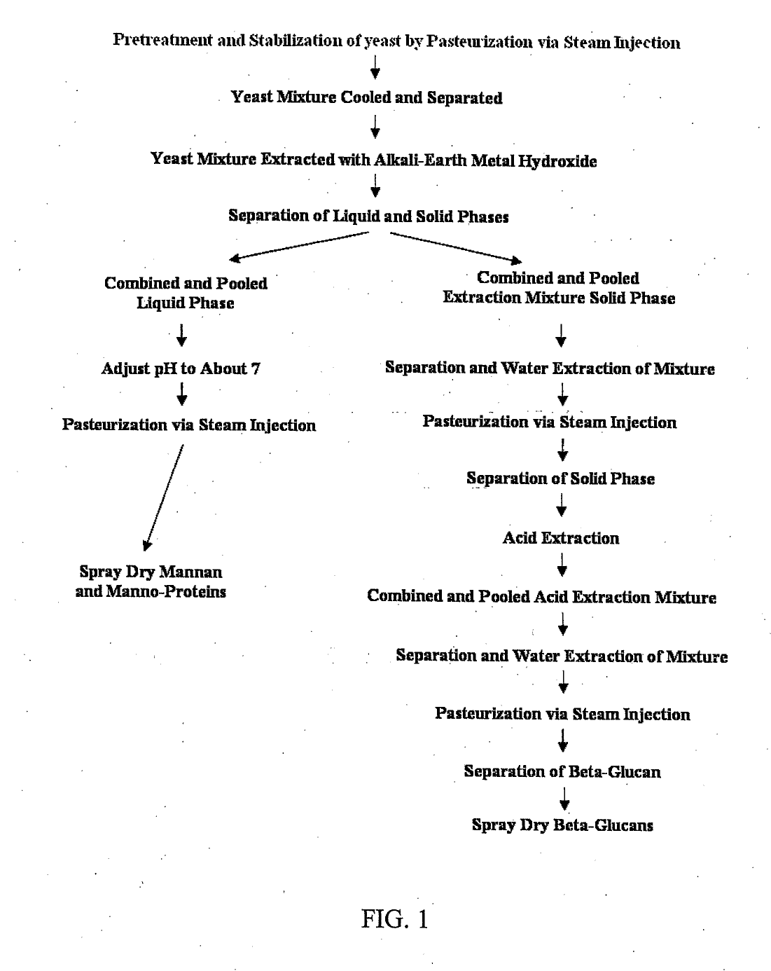

β-(1,3/1,6)-D-glucan was extracted from yeast cells by the following process, which is generally as shown in the flowchart of FIG. 1. A 150 L sample of spent yeast slurry (approximately 15% solids) was pasteurized by steam injection to a temperature of 100° C. for 20 minutes. The mixture was then separated by centrifugation at 1000-3000×g until the liquid and solid phases were separated. The liquid phase was discarded and the yeast solids were re-suspended in 1:5 volumes water (v/v) with stirring for 15 minutes at 20° C. The mixture was then separated by centrifugation, the liquid was discarded and the yeast solids were suspended in 10 volumes (w/v) of 1.5 N NaOH. The mixture was then heated to 80° C. for 45 minutes with stirring, then autoclaved for 30 minutes at 15 psi at 121° C. The mixture was cooled to 50° C. and left to stir at ambient temperature. The solid and liquid phases were separated by centrifugation and collected. The alkali extraction was performed two additional times using the separated yeast solids and the solid phases were combined. The alkali-extracted liquid phases were pooled and retained for further processing, as described in Example 2. The pooled alkali-extracted solid phase was water extracted as described above, then separated by centrifugation. The liquid phase discarded and solids were retained and water extracted as before. After the second water extraction and prior to separation, the solution was pasteurized by steam injection to a temperature of 100° C. for 20 minutes. The mixture was then separated by centrifugation; the liquid phase was discarded and the solids were retained. The solids were subjected to acid extraction with 3% acetic acid in a ratio of 1:10 solids to acid (v/v) to a temperature 80° C. for 1 hour, with stirring. The mixture was separated by centrifugation; the liquid phase was discarded and the solids were retained. The solids were then washed with water, pasteurized, separated as previously described. The solids were then collected and spray dried under the following conditions:

Feed Solids=10.0% (range: 5-25%)

Dry Powder Residual Moisture 8.0% (range: 5-15%)

Inlet Air Temperature=400° F. (204° C.) (range: 400-750° F.)

Outlet Air Temperature=200° F. (93° C.) (range: 200-240° F.)

Feed Atomization using Rotary Atomizer

Dry Powder Cooled to <100° F. using pneumatic cooling/convey system

The composition of the spray-dried material is shown in Table 1.

TABLE 1
Composition of purified β-(1,3/1,6)-D-glucan

| Component | Quantity¹ |
|---|---|
| Carbohydrate | 85.5% |
| Lipid | <12% |
| Protein | 2.87% |
| Moisture | 8.5% |
| Biological Activity | >40 μg Bb released/mg YBG |
| (Alternative Complement) | |

¹results shown are an average of 3 different preparations (Lot Nos. 040816, 040511, 040601).

The biological activity of the β-(1,3/1,6)-D-glucan composition was determined by an in vitro alternative complement activation experiment performed at the National Jewish Medical & Research Center, Denver, Colo.). Briefly, 1 part of a suspension of the β-(1,3/1,6)-D-glucan composition (1 mg/ml, 0.4 mg/ml, and 0.1 mg/ml) is mixed with 9 parts of fresh human serum. After 30 minutes on incubation at 37° C., the mixture is centrifuged to remove insoluble particles. The supernatant is tested for complement activation by quantitatively measuring Bb, a protein fragment released upon activation of the complement protein Factor B. Zymosan 5 mg/ml is used as a control.

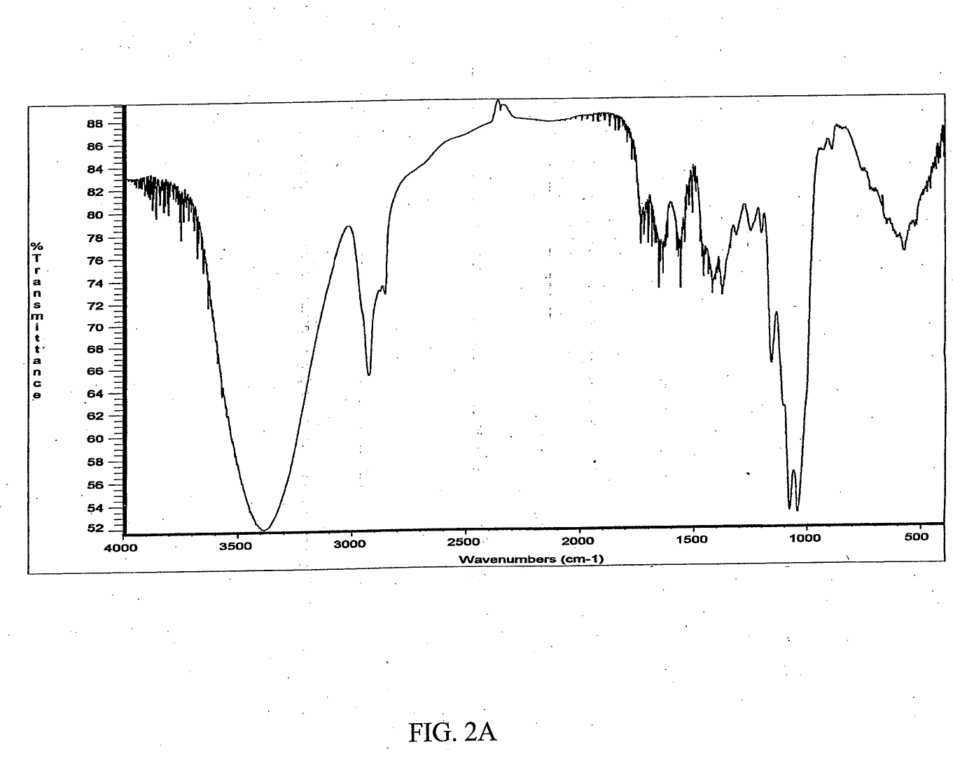

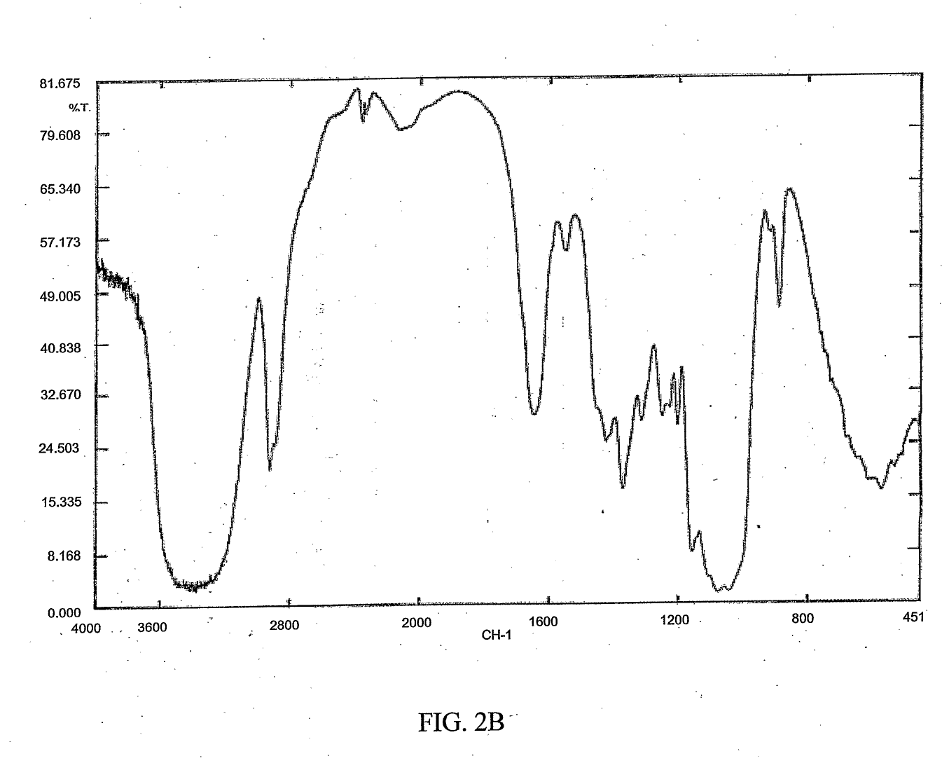

The structural characteristics of the animal grade β-glucan obtained by the above method were compared to those of pharmaceutical grade β-glucan (>90% pure). FIGS. 2A and 2B show the FTIR spectra of pharmaceutical grade yeast β-glucan and the β-glucan obtained by the process of the present invention, respectively. While the scale of the X and Y axes are not identical, it can be determined that similar linkages and/or chemical bonds are present in both the pharmaceutical grade β-glucan and the β-glucan obtained by the process presently described.

The NMR spectra of pharmaceutical grade yeast β-glucan, and the β-glucan obtained by the process of the present invention show similar signals in the 60-140 range (data not shown), and the features responsible for these signals may contribute to the immune bioactivity of β-glucan. The NMR spectrum for the MacroGuard™ product shows a marked absence of these signals (data not shown), which may explain the product's limited bioactivity.

### Example 2

### Separation of Mannan and Manno-Protein Complexes from the Liquid Phase

The alkali-extracted liquid phase retained in Example 1 was further processed, as generally described in the flowchart of FIG. 1. The pH of the liquid was adjusted to 7.0 with HCl. The solution was then pasteurized by steam injection to a temperature of 100° C. for 20 minutes. The mannans and manno-proteins were then isolated from the liquid phase by spray drying under the following conditions:

Feed Solids=10.0% (range: 5-25%)

Dry Powder Residual Moisture=8.0% (range: 5-15%)

Inlet Air Temperature=400° F. (204° C.) (range: 400-750° F.)

Outlet Air Temperature=200° F. (93° C.) (range: 200-240° F.)

Feed Atomiization using Rotary Atomizer

Dry Powder Cooled to <100° F. using pneumatic cooling/convey system

The composition of the dried mannan and manno-protein material is shown in Table 2.

TABLE 2
Composition of mannans and manno-protein complexes

| Component | Quantity¹ |
|---|---|
| Carbohydrate | >30% |
| Lipid | 0.17% |
| Protein | 22% |
| Sulfated Ash | 11% |

¹Lot No. 0410-0531

### Example 3

### Comparative Effects of Various Yeast β-Glucan Compositions

The comparative effects of various yeast β-glucan compositions were determined by macrophage activation of RAW264 macrophage-like cells according to the method of Baggionlini et al. (1986) Methods in Enzymology, 132:395), with some modification. Briefly, BAC or RAW264 target cells were plated into 96 well tissue culture plates and cultured until confluence in phenol red-free alpha-minimum essential medium, supplemented with 10% fetal bovine serum. Thereafter, media were removed, cells washed, and subsequently substrate (homovanlllic acid) and test substances were added. Following an incubation period of 1 hour, the assay reaction was stopped, and the resulting fluorescence measured with an ELISA-reader at max excitation=312 nm, max emission=420 nm. As a positive control, commercial grade Zymosan was used; test substances include pharmaceutical grade β-glucan, the MacroGuard™ product, and the β-glucan produced according to Example 1 (YBG). To determine the amount of H₂O₂ released by cells treated with the various β-glucan compositions, a standard curve (with exogenous H₂O₂ added to the assay) was established.

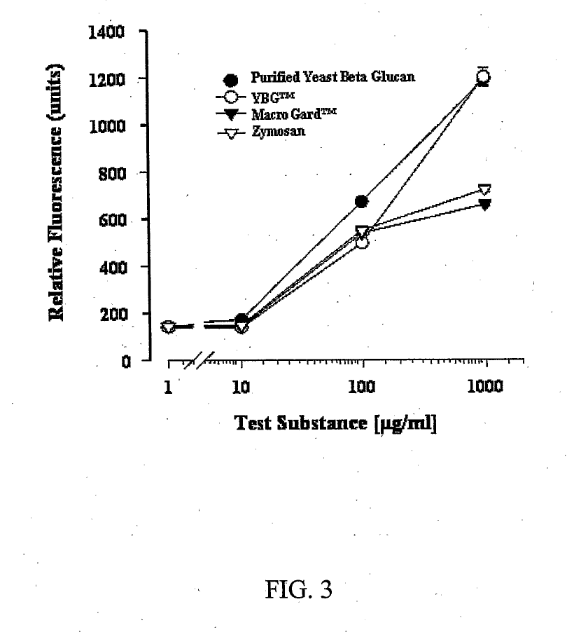

The results for the comparative assay are shown in FIG. 3, in a semi-logarithmic scale. The assay is most useful in a dosage range between 1 to 10 nano moles H₂O₂ release. The effect of the YBG composition is shown to be essentially the same as that of pharmaceutical grade β-glucan, and is more effective than both Zymosan and MacroGuard™.

### Example 4

### Stability of β-(1,3/1,6)-D-Glucan Composition

The stability of the β-glucan produced as described in Example 1 (YBG) was assayed to determine the shelf-life of the product. YBG lot No. 020331AF was tested upon production, after 12 months, after 24 months of storage in a cool (20-25° C.), dry (e.g., free from pooled moisture) place in a sealed container. The results of the tests are shown in Table 3. In addition, the stability of the activity of YBG was determined for 3 different lots of YBG. Activity of the product was measured as described in Example 1 upon production, and after 3, 6, 12 and 24 months of storage in a cool, dry (e.g., free from pooled moisture) place in a sealed and/or plastic lined container. Results of these assays are shown in Table 4.

TABLE 3
Stability of YBG lot No. 020331AF.

| Test Description | Initial | 12 Month | 24 Month |
|---|---|---|---|
| Physical Form | Powder | Powder | Powder |
| Percent Moisture | <10% | <10% | <10% |
| Identification (FTIR) | Pass | Pass | Pass |
| Total Aerobic Plate count | <1000 | <1000 | <1000 |
| (CFU/g) | | | |
| S. aureus | Negative | Negative | Negative |
| E. coli | Negative | Negative | Negative |
| Ps. aeruginose | Negative | Negative | Negative |
| Total Mold & Yeast (CFU/g) | <1000 | <1000 | <1000 |
| Carbohydrate | 87.9% | 87.2% | 86.5% |
| Protein¹ | 2.95% | 2.93% | 2.93% |

¹N × 6.25

TABLE 4
Stability of YBG activity

| Lot No. | Activity¹ | | | | |
|---|---|---|---|---|---|
| (Year produced) | Initial | 3 month | 6 month | 12 month | 24 month |
| 020331AF (2002) | 56.0 | 55.4 | 55.2 | 51.3 | 50.2 |
| 030227AF (2003) | 69.49 | 69.25 | 68.3 | 67.5 | NC² |
| 040601 (2004) | 54.24 | 54.1 | 53.2 | NC | NC |

¹expressed in μg Bb/mg sample
²testing not completed

The results of Tables 3 and 4 indicate that YBG is quite stable for at least 24 months from the date of manufacture when stored in a cool, dry place.

### Example 5

### Use of β-(1,3/1.6)-D-Glucan as an Additive in Swine Feed and Effect Thereof

The effect of using of β-glucan produced as described in Example 1 (YBG) in pig feed was compared to a commercial vaccine and adjuvants. This study, conducted on weaned pigs, compares: growth, health, and response to vaccination, when incremental doses of yeast β-glucan were included in their diets beginning at 3 weeks of age (time of weaning) for the duration of 5 weeks. The study involved 48 pigs, which were housed in pens of two. The pigs were administered one of the following YBG treatments: an injection of saline (control), or an injection of a live Porcine Respiratory Reproductive Syndrome (PRRS) attenuated vaccine with either 0, 40, 80, or 120 g YBG/1000 kg of complete feed. 3 replicates of each treatment were performed. In addition, 12 pigs were administered multiple injections of an aluminum hydroxide adjuvant, and either a single or a double injection of an oil based adjuvant. Blood samples were taken from the anterior vena cava at days 7, 21, and 35 of the study. The PRRS virus (PRRSv) antibodies were quantified using the IDEXX™ PRRS ELISA test kit.

The influence of β-(1,3/1,6)-D-glucan on the growth efficiency and immune response of pigs vaccinated with a PRRSv attenuated vaccine and a saline control is shown in Table 5. The results show that two doses of oil adjuvant and live virus injections negatively impact the growth rate and feed conversion of weaned pigs. β-glucan produced according to the process of Example 1 (YBG) was able to reduce the vaccine-associated growth reduction at a dose of 80 g/1000 kg of complete feed. In addition, YBG was able to increase the antibody response to vaccination when included at 80 g/1000 kg of complete feed. Therefore, YBG is able to boost the immune response of pigs while improving the growth rate, even during an infectious or immune system challenge.

TABLE 5
Effect of YBG on the growth efficiency and immune
response of pigs vaccinated with PRRS vaccine

| Average Daily Growth Rate | g/day | S.E. | |
|---|---|---|---|
| Control | 436 | 21 | a¹ |
| YBG 40 g/1000 kg | 425 | 25 | a |
| YBG 80 g/1000 kg | 390 | 25 | a |
| Al(OH) adjuvant | 397 | 25 | a |
| Oil adjuvant one dose | 415 | 25 | a |
| Oil adjuvant two doses | 275 | 25 | b¹ |
| PPRSv Control @ YBG 0 g/1000 kg | 363 | 22 | c¹ |
| PPRSv @ YBG 40 g/1000 kg | 400 | 21 | a, c |
| PPRSv @ YBG 80 g/1000 kg | 428 | 22 | a |
| PPRSv @ YBG 120 g/1000 kg | 362 | 21 | c |
| Feed Conversion | F:G | S.E. | |
| Control | 1.626 | 0.044 | a |
| YBG 40 g/1000 kg | 1.618 | 0.054 | a |
| YBG 80 g/1000 kg | 1.773 | 0.053 | b |
| YBG 120 g/1000 kg | 1.607 | 0.047 | a |
| Al(OH) adjuvant | 1.735 | 0.053 | b |
| Oil adjuvant one dose | 1.779 | 0.053 | b |
| Oil adjuvant two doses | 2.010 | 0.055 | c |
| PPRSv Control @ YBG 0 g/1000 kg | 1.735 | 0.046 | b |
| PPRSv @ YBG 40 g/1000 kg | 1.655 | 0.045 | a, b |
| PPRSv @ YBG 80 g/1000 kg | 1.716 | 0.046 | a, b |
| PPRSv @ YBG 120 g/1000 kg | 1.653 | 0.045 | a, b |
| Treatment | S/P ratio | S.E. | |
| Control | 1.49 | 0.18 | a |
| PPRSv @ YBG 40 g/1000 kg | 1.62 | 0.18 | a |
| PPRSv @ YBG 80 g/1000 kg | 2.20 | 0.18 | b |
| PPRSv @ YBG 120 g/1000 kg | 1.26 | 0.22 | a |

¹a, b, and c indicate statistically different groups at p < 0.05 (i.e. group “a” is significantly different than group “b” and group “c”, in each category); results with the same letter grouping are not significantly different at p < 0.05.

### Example 6

### Effect of β-(1.3)/(1,6)-D-Glucan Gestating Swine Sows

A study to determine the influence of β-glucan produced according to Example 1 (YBG) on pregnant (gestating) sows and the survival of piglets to weaning was conducted. The study involved a total of 207 sows, 28 days prior to farrowing, divided into 3 groups. The first group (control) was fed a regular diet with no vitamin supplement; the second group was fed regular feed and a vitamin supplement; and the last group was fed YBG at 1 g/sow/day (equivalent to 400 g/1000 kg). All sows were randomly allocated to the farrowing rooms, and farrowed as a batch within approximately 10 days of each other. The piglet mortality rates were measured for the first two weeks of life. Three replicates of this study were performed.

The effect of β-(1,3/1,6)-D-glucan on the gestating sow and the impact on the number of piglets born alive, and the number weaned is shown in Table 6. When the β-glucan produced by this invention was administered to gestating sows at 1 g/sow/day (equivalent to 400 g/1000 kg) the number of piglets born increased by over 10.8% and weaned per sow increased by over 7.3% as compared to the controls. This represents a significant increase (p<0.05) over the control data, and translates into significant increase in productivity, savings and cost benefit ratio for swine producers.

TABLE 6
Effect of TBG on litter size and survival

| Treatment | Average Piglets Born Alive | | Average Piglet Weaned | |
|---|---|---|---|---|
| Control | 10.35 | a¹ | 9.27 | a |
| Beta Glucan | 11.47 | b¹ | 9.96 | b |
| Vitamin premix | 11.15 | a, b | 9.32 | a |

¹a and b indicate statistically different groups at p < 0.05 (i.e. group “a” is significantly different than group “b” in each category); results with the same letter grouping are not significantly different at p < 0.05.

### Example 7

### Effect of β-(13/1,6)-D-Glucan on Colostrum Quality of Swine

A study was conducted to determine whether feeding β-glucan produced according to Example 1 (YBG) to pregnant sows would improve the passive transfer of anti-Mycoplasma antibodies to the piglets. 150 pigs were sampled from sows fed either 0, 0.5, or 1.0 g of YBG/sow/day as a top dress for 4 weeks prior to farrowing. All sows were vaccinated with a commercial oil-adjuvant Mycoplasma hyopneuminiae (Boehringer Ingelheim, Canada) 14 days prior to farrowing, and piglets were sampled 18 days after birth. Antibody titers were measured with a commercial (DAKOT™) ELISA test kit. The data was analyzed using a mixed model regression controlling for litter size, birth weight and parity as a random effect. The results are shown in Table 7.

TABLE 7
Effect of YBG on passive transfer of antibodies

| Treatment | Mean Titer | |
|---|---|---|
| Control | 53.2 | a¹ |
| YBG 0.5 g/day | 75.7 | a |
| YBG 1.0 g/day | 137.7 | b¹ |

¹a and b indicate statistically different groups at p < 0.05 (i.e. group “a” is significantly different than group “b”, in each category); results with the same letter grouping are not significantly different at p < 0.05.

In general, sows fed YBG pre-farrow at prescribed rates and period were shown to enhance colostrum quality (i.e., increase antibody titres) and thus increase disease protection, and therefore survival, in piglets. Specifically, the results show that a dose of 1.0 g YBG/sow/day significantly increases the maternal/passive transfer of anti-Mycoplasma antibodies to piglets, but a dose of 0.5 g YBG/sow/day shows no significant effect over control group. Thus, the immune stimulation of pregnant sows with YBG may improve passive transfer of immunoglobulins to piglets, which in turn may lead to improved protection to infections and increased growth and productivity of the piglets. YBG has the net effect of enhancing the passive immunity and disease protection in young immune compromised piglets via maternal antibodies.

### Example 8

### Effect of β-(1,3/1.6)-D-glucan on the Growth of Broiler Chickens

A study was conducted to determine the effect of O-glucan produced according to Example 1 (YBG) on the growth of broiler chickens. The study was performed on farms in Nova Scotia, Canada. Approximately 6300 chickens were housed on the bottom tier of a poultry house and fed a diet containing 40 g YBG/1000 kg of complete feed for 2 weeks, followed by 20 g YBG/1000 kg of complete feed for 4 weeks, while another approximately 6300 chickens were housed on the top tier of the same poultry house and fed a diet containing the growth promotion antibiotic Stafac™ (Phibro Animal Health Ltd., ON Canada). A total of four independent placements were made through the same barn under similar conditions. All feed in both the YBG and Stafac™ groups was supplemented with the coccidiostat Coban™ (Elanco Animal Health, Guelph, ON Canada). At the end of the six weeks, performance was assessed using the following criteria: mortality, final weight, average daily gain, feed conversion, and condemnation rate. The results of this experiment, summarized in Table 8 below, show comparable growth parameters in chickens fed on the two feed regimens, indicating that it is feasible to farm chickens without growth promotion antibiotics.

TABLE 8
Effect of YBG compared to antibiotics on growth of chickens

| Criteria | YBG + Coban ™ | Stafac ™ + Coban ™ |
|---|---|---|
| Total Number of Birds | 25,431 | 25,564 |
| placed¹ | | |
| Mortality (%) | 1.63% | 1.81% |
| Weight (kg) | 2.03 | 2.02 |
| Age (days) | 40.42 | 40.42 |
| Average Daily Gain (g/day) | 50.59 | 50.69 |
| Producer Condemnation % | 0.65% | 0.65% |
| Feed Conversion² | 1.82 | 1.78 |

¹Table summarizes a total of four (4) independent commercial placements of approximately 6300 chicks/trial/treatment
²Feed conversions are based on the ratio of food consumed to mass of bird. Typical commercial ratios range from 1.5-1.9 kg of feed to 1 kg of weight gain.

### Example 9

### Effect of β-(1,3/1,6)-D-Glucan on Immune Parameters in Broiler Chickens

The effectiveness of β-(1,3/1,6)-D-glucan produced according to Example 1 (YBG) as an immune enhancer was assayed. Blood samples were taken from chickens fed a diet containing 40 g YBG/1000 kg of complete feed for the first 2 weeks, followed by 20 g YBG/1000 kg complete feed for the last 4 weeks, as well as from chickens fed a traditional diet containing growth promotion antibiotics. The blood samples were analyzed using a proprietary lymphocyte proliferative capacity assay developed by PharmaGap Inc. (Ontario Canada) in the presence of the following blastogenic substances: Concanavalin A (ConA), phytohemaglutin (PHA), phorbol myristate acetate (PMA)+Ionomycin, lipopolysaccharide (LPS)+dextran sulfate (DxS), and poke weed mnitogen (PWM. Results are shown in Tables 9 and 10.

TABLE 9
Effect of antibiotic treatment on lymphocyte proliferation in
chickens¹

| | C2 | C3 | C5 | C7 | C8 | C10 | C11 | C13 | C17 | C19 | C20 | C22 | Avg. |
|---|---|---|---|---|---|---|---|---|---|---|---|---|---|
| Control | 100 | 100 | 100 | 100 | 100 | 100 | 100 | 100 | 100 | 100 | 100 | 100 | 100 |
| ConA | 128 | 95 | 95 | 95 | 97 | 87 | 110 | 92 | 100 | 103 | 97 | 92 | 99 |
| PHA | 72 | 86 | 56 | 69 | 85 | 94 | 91 | 94 | 83 | 106 | 74 | 81 | 83 |
| PWM | 81 | 88 | 106 | 85 | 89 | 73 | 84 | 66 | 92 | 106 | 86 | 104 | 88 |
| LPS + DxS | 100 | 126 | 165 | 100 | 100 | 103 | 139 | 63 | 118 | 133 | 83 | 102 | 111 |
| PMA + Iono | 106 | 103 | 147 | 136 | 97 | 96 | 178 | 53 | 102 | 117 | 89 | 104 | 111 |

¹Increase in cell number upon stimulation, expressed as percentage of unstimulated matching controls for chickens fed a feed containing a growth promotion antibiotic.

Table 9 shows that in animals fed a diet containing growth promotion antibiotics, the response of lymphocytes to these various substances was heterogeneous with a significantly low stimulation of immune cells. The response to ConA, PWM, and LPS was either not significantly different from the control and in some animals the response was significantly less than in the control (e.g., response to PHA in animals Nos. 2, 5, 7, and 20; response to PMA in animal 13; response to PWM in animals 13 and 20, response to LPS in animal 13.). Thus, animals fed with only growth promotion antibiotics have an unactivated immune system that would potentially be more susceptible to bacterial and moreover to viral infections that the antibiotic does not treat. Furthermore, antibiotics have no effects on treating or resisting viral infections.

TABLE 10
Effect of YBG treatment on lymphocyte proliferation in
chickens¹

| | C1 | C4 | C6 | C9 | C14 | C15 | C16 | C18 | C21 | C24 | Avg. |
|---|---|---|---|---|---|---|---|---|---|---|---|
| Control | 100 | 100 | 100 | 100 | 100 | 100 | 100 | 100 | 100 | 100 | 100 |
| Con A | 163 | 133 | 134 | 114 | 116 | 113 | 107 | 120 | 107 | 98 | 121 |
| PHA | 173 | 95 | 112 | 121 | 108 | 122 | 127 | 100 | 127 | 119 | 120 |
| PWM | 147 | 128 | 149 | 121 | 112 | 101 | 116 | 106 | 113 | 164 | 126 |
| LPS + DxS | 139 | 148 | 159 | 118 | 148 | 123 | 147 | 120 | 116 | 152 | 137 |
| PMA + Iono | 173 | 129 | 143 | 100 | 130 | 125 | 126 | 120 | 108 | 160 | 131 |

¹Increase in cell number upon stimulation, expressed as percentage of unstimulated matching controls for chickens fed a feed containing a growth promotion antibiotic.

In contrast, Table 10 shows that in animals fed a diet containing YBG, the response of lymphocytes to the various substances was significantly enhanced since an increase in lymphocytes proliferation was observed over the unstimulated controls. The response was particularly elevated following PMA and LPS stimulation. These data are indicative of an enhanced immune competence and increased resistance to both bacterial and viral infections in animals fed YBG.

### Example 10

### Effect of β-(1,3/1,6)-D-Glucan on Growth Performance and Organ Weights in Broiler Chickens

A study was conducted to determine the impact of β-(1,3/1,6)-D-glucan produced according to Example 1 (YBG) on immune system components compared to growth promotion antibiotics. Three trials were conducted with 912 day-old chicks for each trial. Chicks were randomly assigned to 24 pens (38 birds/pen) and one of three dietary treatments: no growth promotant (control), YBG or virginiamycin. Starter YBG diets contained 40 g YBG/1000 kg of complete feed and the grower and finisher diets contained 20 g YBG/1000 kg of complete feed. The birds were fed a starter diet from 0 to 14 days (d), a grower diet from 14 to 24 days and a finisher diet from 24 to 38 days. All birds were manually weighed on days 0, 14, 24, and 38 and feed consumed was monitored throughout the study. At 14 and 38 days of age, 48 broilers (2/pen) were euthanized and the spleen and bursa of Fabricius removed and weighed. Blood samples from 21 and 35 days of age were fixed to slides for differential staining.

Organ weights as a proportion of body weight and white blood cell counts were the same between treatment groups. The feed efficiency for each dietary treatments were also the same throughout the rearing period. On average, birds given antibiotics were larger (818 g) than birds given YBG (771 g) and controls (752 g) by day 24. However, by day 38 the birds given YBG were no longer significantly smaller (1987 g) than the antibiotic group (2009 g) at p<0.05. The control group showed smaller average body weights (1934 g; p>0.05) than the other two treatments at the end of the growth period. These results indicate that YBG is as effective in promoting growth of broiler chickens as a commonly used antibiotic. Therefore, the replacement of growth promotion antibiotics by YBG is feasible.

### Example 11

### Effect of β-(1.3/1.6)-D-Glucan on Growth of Broiler Chickens

A study was conducted to determine the effect of β-glucan produced according to Example 1 (YBG) on the growth of broiler chickens. A total of 900 broiler chickens were fed a diet containing either no growth promotant (control), a growth promotion antibiotic, 20 g YBG/1000 kg of complete feed, or 40 g YBG/1000 kg of complete feed for 6 weeks. The results of this experiment are summarized in Table 12 below.

TABLE 11
Effects of YBG on the productive performance of broiler chickens
Weeks 0-3

| Treatment | Daily gain (g) | | Food intake (g) | | Feed conversion | | Weight on day 21 (kg) | |
|---|---|---|---|---|---|---|---|---|
| Control | 27.49 ± 1.16 | b¹ | 47.27 ± 1.65 | a | 1.72 ± 0.06 | a | 0.634 ± 0.02 | b |
| Antibiotics | 29.04 ± 1.86 | a¹ | 48.55 ± 2.30 | a | 1.67 ± 0.05 | b | 0.658 ± 0.04 | a |
| 20 g/1000 kg | 27.66 ± 1.46 | b | 47.75 ± 2.34 | a | 1.73 ± 0.07 | a | 0.629 ± 0.03 | b |
| 40 g/1000 kg | 27.77 ± 1.26 | b | 48.06 ± 2.68 | a | 1.73 ± 0.06 | a | 0.632 ± 0.03 | b |

Weeks 4-6

| | | | | | | | Weight at week | | Mortality |
|---|---|---|---|---|---|---|---|---|---|
| Treatment | Daily gain (g) | | Food intake (g) | | Feed conversion | | 6 (kg) | | (%) |
| Control | 63.46 ± 3.34 | b | 134.99 ± 5.09 | a | 2.13 ± 0.09 | a | 1.962 ± 0.07 | b | 4.46 |
| Antibiotics | 65.32 ± 3.49 | a, b | 134.36 ± 10.43 | a | 2.06 ± 0.16 | a | 2.031 ± 0.10 | a | 3.12 |
| 20 g/1000 kg | 63.91 ± 2.34 | a, b | 135.29 ± 5.74 | a | 2.12 ± 0.07 | a | 1.976 ± 0.06 | b | 2.68 |
| 40 g/1000 kg | 65.59 ± 2.79 | a | 138.44 ± 12.27 | a | 2.10 ± 0.12 | a | 2.01 ± 0.05 | a, b | 3.27 |

¹a and b indicate statistically different groups at p < 0.05 (i.e. group “a” is significantly different than group “b”, in each category); results with the same letter grouping are not significantly different at p < 0.05.

Compared to the control group, the YBG 20 g/kg and 40 g/kg groups show no difference in daily gain, food intake, feed conversion or weight over weeks 0-3. However, the antibiotics group shows a significant difference on the daily gain, feed conversion and weight, but no difference on food intake. The results suggest that the effect of YBG is slower than that of the antibiotics in the first 3 weeks. In contrast, by week 6, the daily gain observed in chickens fed 40 g YBG/1000 kg complete feed is the highest among the different treatment groups. The chickens fed 40 g YBG/1000 kg complete feed also show an average weight similar to the antibiotic treated chickens. In addition, a trend of low mortality among YBG fed chickens was observed. In summary, the results show comparable growth parameters in chickens fed on the two feed regimens, indicating that it is feasible to farm chickens without growth promotion antibiotics.

### Example 12

### Growth Comparison Between YBG and Antibiotic Fed Turkeys

A study was conducted to determine the effect of O-glucan produced according to Example 1 (YBG) on the growth of turkeys. Traditionally grown turkeys were fed the growth promotion antibiotic Stafac™ at 22 g/1000 kg of complete feed for up to 12 weeks. The YBG grown birds were fed YBG at 40 g/1000 kg of complete feed for the first 6 weeks, followed by YBG at 20 g/1000 kg for the remainder of the growth period (i.e., 5 weeks). In both control and YBG fed diets, the anti-coccidiostat Coban™ was used at a dosage of 22 g/1000 kg of complete feed throughout. Results are shown in Table 11.

TABLE 12
Effect of YBG compared to antibiotics on growth of turkeys

| | YBG | Traditional Grown |
|---|---|---|
| Producer Grade A's | 92-94% | 89% |
| Producer Condemnations | 0.6% | 0.5% |
| Age | 67-75 days | 65-84 |

The response in turkeys was an increase from the typical 85%-90% “Grade A” birds to >92% “Grade A” birds.

### Example 13

### Effect of β-(1,3/1,6)-D-Glucan on Productivity and Survival of Shrimp

A study is conducted in partnership with Kasetsart University, Thailand and the Atlantic Veterinarian College (AVC), PEI Canada to determine the effect of β-glucan produced according to Example 1 (YBG) on the productivity and survival of farmed shrimp. Approximately three million shrimp (Penaeus monodon) are purchased from a nursery, and are obtained from the same batch. The shrimp are divided into 10 ponds, with treatment ponds (5) and control ponds (5) matched for size and shape. Fifty PL shrimp from each nursery batch are initially sampled by PCR and RT-PCR for White Spot Syndrome Virus (WSSV), Infectious Hypodermal and Hematopoietic Necrosis (IHHNV), Penaeus monodon-type baculovirus (MBV), Hepatopancreatic Parvovirus (HPV), Taura Syndrome Virus (TSV) and Yellow Head Virus (YHV) to ensure no disease is present.

YBG is added to the feed for the treatment ponds at a level of 100 g/1000 kg of complete feed according to a standard feed regime designed for each age range of shrimp. The control ponds are fed the same feed, according to the same regime as the research ponds, in the absence of YBG. After 120 days, the shrimp are harvested and the following parameters are measured: feed conversion rate (FCR), average daily gain (ADG), survival rate, yield per acre and count per pound (i.e., sizing). At the end of the trial, a representative samples from each batch/pond is also sampled and examined by PCR and RT-PCR for WSSV, IHHNV, MBV, HPV, TSV and YHV.

The YBG fed group has a higher resistance to viral and bacterial disease, and is thus better able to cope with those diseases present. As a result, overall growth performance is enhanced, depending on the actual disease level the control and YBG feed shrimp are subjected to during the trial. Also, the survival rate for the YBG shrimp is increased by at least 10% over the controls, with similar gains in average daily gain, yield per acre and feed conversion, depending on disease level in ponds.

The present invention has been described with regard to one or more embodiments. However, it will be apparent to persons skilled in the art that a number of variations and modifications can be made without departing from the scope of the invention as defined in the claims.

The contents of all references and patents cited throughout this application are hereby incorporated by reference in their entirety.

1-32. (canceled)

33. A process for producing β-(1,3/1,6)-D-glucan from a cellular source, said process comprising:
a) alkali extraction of the cellular source;
b) water extraction;
c) acid extraction; and
d) water extraction, to produce a solid component comprising at least about 70% β-(1,3/1,6)-D-glucan by dry weight,
wherein at least one step of water extraction includes pasteurization by steam injection to a temperature of about 100° C. for a time in the range of about 15 to about 30 minutes.

34. The process according to claim 33, wherein both steps of water extraction include pasteurization.

35. The process according to claim 33, wherein the alkali extraction step (step a)) comprises treating the cellular source with an alkali solution, and heating to a temperature in the range of about 45° C. to about 80° C. for about 30 minutes, followed by an increase in temperature to a temperature in the range of about 95° C. to about 150° C. for a time in the range of about 15 minutes to about 120 minutes at a pressure in the range of about 1 psi to about 25 psi.

36. The process according to claim 35, wherein the alkali extraction step comprises heating to a temperature of about 60° C. for about 45 minutes, followed by an increase in temperature to about 121° C. for about 30 minutes at a pressure in the range of about 1 psi to about 25 psi.

37. The process according to claim 35, wherein the alkali solution is an alkali-metal hydroxide or alkali-earth metal hydroxide solution in a ratio in the range of about 1:5 to 1:15 cellular source to alkali solution.

38. The process according to claim 33, wherein the water extraction step (steps b) and d)), water is added at a ratio in the range of about 1:4 to about 1:20 solids to water, for a time in the range of about 15 minutes to about 2.5 hours at a temperature in the range of about 20° C. to about 100° C.

39. The process according to claim 33, wherein the acid extraction step (step c)) comprises treating with an acid solution at a ratio in the range of about 1:4 to about 1:20 solids to acid solution, and is heated to a temperature in the range of about 45° C. to about 120° C. for a time in the range of about 15 minutes to about 2 hours.

40. The process according to claim 33, wherein each of steps a) to d) is followed by a step of separating the treated material into a liquid phase and a solid phase, and wherein each subsequent step is performed on the solid phase.

41. The process according to claim 40, wherein the sequence of steps a) through b) is performed 1, 2, or 3 times.

42. The process according to claim 40, wherein the sequence of steps c) through d) is performed 1, 2, or 3 times.

43. The process according to claim 33, wherein the liquid phase of step a) is collected and combined.

44. The process according to claim 33, wherein step a) is preceded by pasteurization of the cellular source by steam injection to a temperature of about 100° C. for a time in the range of about 15 to about 30 minutes.

45. The process according to claim 33, wherein the cellular source is selected from the group consisting of Baker's yeast, Brewer's yeast, spent yeast, and yeast cell wall material.

46. A process for producing β-(1,3/1,6)-D-glucan from yeast, said process comprising:
a) pasteurization of the yeast by steam injection to a temperature of about 100° C. for a time in the range of about 15 to about 30 minutes;
b) separation of the pasteurized yeast into a first liquid phase and a first solid phase;
c) alkali extraction of the first solid phase with an alkali-metal or alkali-earth metal hydroxide solution in a ratio in the range of about 1:5 to about 1:15 solids to alkali solution, and heating to a temperature in the range of about 45° C. to about 80° C. for about 30 minutes;
d) increasing the temperature to a temperature in the range of about 95° C. to about 150° C. for a time in the range of about 15 min to about 120 min at a pressure in the range of about 1 psi to about 25 psi, to form an alkali-extracted mixture;
e) separation of the alkali-extracted mixture into a second liquid phase and a second solid phase;
f) water extraction of the second solid phase with water at a ratio in the range of about 1:4 to about 1:20 solids to water, for a time in the range of about 15 minutes to about 2.5 hours at a temperature in the range of about 20° C. to about 100° C., to form a water-extracted mixture;
g) pasteurization of the water-extracted mixture by steam injection to a temperature of about 100° C. for a time in the range of about 15 to about 30 minutes;
h) separation of the water-extracted mixture into a third liquid phase and a third solid phase;
i) acid extraction of the third solid phase with an acid solution in a ratio in the range of about 1:4 to about 1:20 solids to acid solution and heating to a temperature in the range of about 45° C. to about 120° C. for a time in the range of about 15 minutes to about 2 hours, to form an acid-extracted mixture;
j) separation of the acid-extracted mixture into a fourth liquid phase and a fourth solid phase;
k) water extraction of the fourth solid phase with water at a ratio in the range of about 1:4 to about 1:20 solids to water, for a time in the range of about 15 minutes to about 2.5 hours at a temperature in the range of about 20° C. to about 100° C., to form a water-extracted mixture;
l) pasteurization of the water-extracted mixture by steam injection to a temperature of about 100° C. for a time in the range of about 15 to about 30 minutes; and
m) separation of the water-extracted mixture into a fifth liquid phase and a fifth solid phase, the fifth solid phase comprising at least about 70% β-(1,3/1,6)-D-glucan by dry weight.

47. The process according to claim 46, wherein the sequence of steps a) through e) is performed 1, 2, or 3 times.

48. The process according to claim 46, wherein the sequence of steps i) through m) is performed 1, 2, or 3 times.

49. The process according to claim 33, further comprising producing mannan and manno-protein complexes by:
i) collecting a liquid phase obtained in one or more than one alkali extraction step (step a));
ii) adjusting the pH of the liquid phase of step i) to a pH in the range of about 5.0 to about 8.0 with an acid;
iii) pasteurizing the liquid phase of step ii) by steam injection to a temperature of about 100° C. for a time in the range of about 15 to about 30 minutes; and
iv) isolating the mannan and manno-protein complexes from the pasteurized liquid phase of step iii).

50. The process according to claim 46, further comprising producing mannan and manno-protein by:
i) collecting the second liquid phases obtained in one or more than one alkali extraction step (step e));
ii) adjusting the pH of the liquid phase of step i) to a pH in the range of about 5.0 to about 8.0 with an acid;
iii) pasteurizing the liquid phase of step ii) by steam injection to a temperature of about 100° C. for a time in the range of about 15 to about 30 minutes; and
iv) isolating the mannan and manno-protein complexes from the pasteurized liquid phase of step iii).

51. The process according to claim 49, wherein the step of isolating (step iv) is by precipitation and centrifugation, or by drying.

52. The process according to claim 49, wherein the solids from iv) will comprise at least about 30% mannan and manno-protein complexes.

53. The process according to claim 52, wherein the solids from iv) will comprise at least about 5% protein.

54. The process according to claim 50, wherein the step of isolating (step iv) is by precipitation and centrifugation, or by drying.

55. The process according to claim 50, wherein the solids from iv) will comprise at least about 30% mannan and manno-protein complexes.

56. The process according to claim 55, wherein the solids from iv) will comprise at least about 5% protein.

57. A β-(1,3/1,6)-D-glucan composition prepared by the process according to claim 33.

58. An animal feed comprising the β-(1,3/1,6)-D-glucan composition of claim 57 in an amount effective for enhancing immuno-competence of an animal.

59. A β-(1,3/1,6)-D-glucan composition prepared by the process according to claim 46.

60. An animal feed comprising the β-(1,3/1,6)-D-glucan composition of claim 59 in an amount effective for enhancing immuno-competence of an animal.

61. A β-(1,3/1,6)-D-glucan composition comprising at least about 70% β-(1,3/1,6)-D-glucan, wherein the composition has a biological activity of at least 30 μg Bb released per mg of β-(1,3/1,6)-D-glucan.

62. An animal feed comprising the β-(1,3/1,6)-D-glucan composition of claim 61 in an amount effective for enhancing immuno-competence of an animal.

63. The animal feed according to claim 57, wherein the animal feed is for an animal selected from the group consisting of poultry, swine, equine species, cattle, companion animals, and aquaculture species.

64. The animal feed according to claim 57, wherein the effective amount is a concentration in the range of about 5 g/1000 kg to about 500 g/1000 kg of the feed.

65. The animal feed according to claim 57, wherein the animal is poultry, and the effective amount is in the range of about 20 g/1000 kg to about 50 g/1000 kg of feed.

66. The animal feed according to claim 57, wherein the animal is swine, and the effective amount is in the range of about 20 g/1000 kg to about 500 g/1000 kg of feed, based on swine growth cycle and duration of use.

67. The animal feed according to claim 57, wherein the animal is an equine species, and the effective amount is in the range of about 25 g/1000 kg to about 300 g/1000 kg of feed.

68. The animal feed according to claim 57, wherein the animal is an aquaculture species, and the effective amount is in the range of about 35 g/1000 kg to about 300 g/1000 kg.

69. An animal feed comprising:
a) β-(1,3/1,6)-D-glucan composition prepared by a process comprising:
i) alkali extraction of the cellular source;
ii) water extraction;
iii) acid extraction; and
iv) water extraction, to produce a solid component comprising at least about 70% β-(1,3/1,6)-D-glucan by dry weight,
wherein at least one step of water extraction includes pasteurization by steam injection to a temperature of about 100° C. for a time in the range of about 15 to about 30 minutes and wherein said β-(1,3/1,6)-D-glucan composition is in an amount effective for enhancing immuno-competence of the animals; and
b) mannans and manno-proteins produced by process according to claim 49 in an amount sufficient to inhibit bacterial adhesion to the intestinal walls of animals.

70. The animal feed of claim 69, wherein the amount of β-(1,3/1,6)-D-glucan is in the animal feed is in the range of about 5 g/1000 kg to about 500 g/1000 kg of complete feed, and the amount of mannans and/or manno-proteins in the animal feed is in the range of about 100 g/1000 kg to about 4000 g/1000 kg of complete feed.

71. An animal feed comprising:
a) β-(1,3/1,6)-D-glucan composition prepared by a process comprising:
i) alkali extraction of the cellular source;
ii) water extraction;
iii) acid extraction; and
iv) water extraction, to produce a solid component comprising at least about 70% β-(1,3/1,6)-D-glucan by dry weight,
wherein at least one step of water extraction includes pasteurization by steam injection to a temperature of about 100° C. for a time in the range of about 15 to about 30 minutes and wherein said β-(1,3/1,6)-D-glucan composition is in an amount effective for enhancing immuno-competence of the animals; and
b) mannans and manno-proteins produced by process according to claim 50 in an amount sufficient to inhibit bacterial adhesion to the intestinal walls of animals.

72. The animal feed of claim 71, wherein the amount of β-(1,3/1,6)-D-glucan is in the animal feed is in the range of about 5 g/1000 kg to about 500 g/1000 kg of complete feed, and the amount of mannans and/or manno-proteins in the animal feed is in the range of about 100 g/1000 kg to about 4000 g/1000 kg of complete feed.

73. An animal feed comprising:
a) β-(1,3/1,6)-D-glucan composition prepared by a process comprising:
i) pasteurization of the yeast by steam injection to a temperature of about 100° C. for a time in the range of about 15 to about 30 minutes;
ii) separation of the pasteurized yeast into a first liquid phase and a first solid phase;
iii) alkali extraction of the first solid phase with an alkali-metal or alkali-earth metal hydroxide solution in a ratio in the range of about 1:5 to about 1:15 solids to alkali solution, and heating to a temperature in the range of about 45° C. to about 80° C. for about 30 minutes;
iv) increasing the temperature to a temperature in the range of about 95° C. to about 150° C. for a time in the range of about 15 min to about 120 min at a pressure in the range of about 1 psi to about 25 psi, to form an alkali-extracted mixture;
v) separation of the alkali-extracted mixture into a second liquid phase and a second solid phase;
vi) water extraction of the second solid phase with water at a ratio in the range of about 1:4 to about 1:20 solids to water, for a time in the range of about 15 minutes to about 2.5 hours at a temperature in the range of about 20° C. to about 100° C., to form a water-extracted mixture;
vii) pasteurization of the water-extracted mixture by steam injection to a temperature of about 100° C. for a time in the range of about 15 to about 30 minutes;
viii) separation of the water-extracted mixture into a third liquid phase and a third solid phase;
ix) acid extraction of the third solid phase with an acid solution in a ratio in the range of about 1:4 to about 1:20 solids to acid solution and heating to a temperature in the range of about 45° C. to about 120° C. for a time in the range of about 15 minutes to about 2 hours, to form an acid-extracted mixture;
x) separation of the acid-extracted mixture into a fourth liquid phase and a fourth solid phase;
xi) water extraction of the fourth solid phase with water at a ratio in the range of about 1:4 to about 1:20 solids to water, for a time in the range of about 15 minutes to about 2.5 hours at a temperature in the range of about 20° C. to about 100° C., to form a water-extracted mixture;
xii) pasteurization of the water-extracted mixture by steam injection to a temperature of about 100° C. for a time in the range of about 15 to about 30 minutes; and
xiii) separation of the water-extracted mixture into a fifth liquid phase and a fifth solid phase, the fifth solid phase comprising at least about 70% β-(1,3/1,6)-D-glucan by dry weight
wherein said β-(1,3/1,6)-D-glucan composition is in an amount effective for enhancing immuno-competence of the animals; and
b) mannans and manno-proteins produced by process according to claim 49 in an amount sufficient to inhibit bacterial adhesion to the intestinal walls of animals.

74. The animal feed of claim 73, wherein the amount of β-(1,3/1,6)-D-glucan is in the animal feed is in the range of about 5 g/1000 kg to about 500 g/1000 kg of complete feed, and the amount of mannans and/or manno-proteins in the animal feed is in the range of about 100 g/1000 kg to about 4000 g/1000 kg of complete feed.

75. An animal feed comprising:
a) β-(1,3/1,6)-D-glucan composition prepared by a process comprising:
i) pasteurization of the yeast by steam injection to a temperature of about 100° C. for a time in the range of about 15 to about 30 minutes;
ii) separation of the pasteurized yeast into a first liquid phase and a first solid phase;
iii) alkali extraction of the first solid phase with an alkali-metal or alkali-earth metal hydroxide solution in a ratio in the range of about 1:5 to about 1:15 solids to alkali solution, and heating to a temperature in the range of about 45° C. to about 80° C. for about 30 minutes;
iv) increasing the temperature to a temperature in the range of about 95° C. to about 150° C. for a time in the range of about 15 min to about 120 min at a pressure in the range of about 1 psi to about 25 psi, to form an alkali-extracted mixture;
v) separation of the alkali-extracted mixture into a second liquid phase and a second solid phase;
vi) water extraction of the second solid phase with water at a ratio in the range of about 1:4 to about 1:20 solids to water, for a time in the range of about 15 minutes to about 2.5 hours at a temperature in the range of about 20° C. to about 100° C., to form a water-extracted mixture;
vii) pasteurization of the water-extracted mixture by steam injection to a temperature of about 100° C. for a time in the range of about 15 to about 30 minutes;
viii) separation of the water-extracted mixture into a third liquid phase and a third solid phase;
ix) acid extraction of the third solid phase with an acid solution in a ratio in the range of about 1:4 to about 1:20 solids to acid solution and heating to a temperature in the range of about 45° C. to about 120° C. for a time in the range of about 15 minutes to about 2 hours, to form an acid-extracted mixture;
x) separation of the acid-extracted mixture into a fourth liquid phase and a fourth solid phase;
xi) water extraction of the fourth solid phase with water at a ratio in the range of about 1:4 to about 1:20 solids to water, for a time in the range of about 15 minutes to about 2.5 hours at a temperature in the range of about 20° C. to about 100° C., to form a water-extracted mixture;
xii) pasteurization of the water-extracted mixture by steam injection to a temperature of about 100° C. for a time in the range of about 15 to about 30 minutes; and
xiii) separation of the water-extracted mixture into a fifth liquid phase and a fifth solid phase, the fifth solid phase comprising at least about 70% β-(1,3/1,6)-D-glucan by dry weight
wherein said β-(1,3/1,6)-D-glucan composition is in an amount effective for enhancing immuno-competence of the animals; and
b) mannans and manno-proteins produced by process according to claim 50 in an amount sufficient to inhibit bacterial adhesion to the intestinal walls of animals.

76. The animal feed of claim 75, wherein the amount of D-(1,3/1,6)-D-glucan is in the animal feed is in the range of about 5 g/1000 kg to about 500 g/1000 kg of complete feed, and the amount of mannans and/or manno-proteins in the animal feed is in the range of about 100 g/1000 kg to about 4000 g/1000 kg of complete feed.

77. An animal feed comprising:
a) β-(1,3/1,6)-D-glucan composition comprising at least about 70% β-(1,3/1,6)-D-glucan, wherein the composition has a biological activity of at least 30 μg Bb released per mg of β-(1,3/1,6)-D-glucan, wherein the β-(1,3/1,6)-D-glucan composition is in an amount effective for enhancing immuno-competence of the animals; and
b) mannans and manno-proteins produced by process according to claim 49 in an amount sufficient to inhibit bacterial adhesion to the intestinal walls of animals.

78. The animal feed of claim 77, wherein the amount of β-(1,3/1,6)-D-glucan is in the animal feed is in the range of about 5 g/1000 kg to about 500 g/1000 kg of complete feed, and the amount of mannans and/or manno-proteins in the animal feed is in the range of about 100 g/1000 kg to about 4000 g/1000 kg of complete feed.

79. An animal feed comprising:
a) β-(1,3/1,6)-D-glucan composition comprising at least about 70% β-(1,3/1,6)-D-glucan, wherein the composition has a biological activity of at least 30 μg Bb released per mg of β-(1,3/1,6)-D-glucan, wherein the β-(1,3/1,6)-D-glucan composition is in an amount effective for enhancing immuno-competence of the animals; and
b) mannans and manno-proteins produced by process according to claim 50 in an amount sufficient to inhibit bacterial adhesion to the intestinal walls of animals.

80. The animal feed of claim 79, wherein the amount of β-(1,3/1,6)-D-glucan is in the animal feed is in the range of about 5 g/1000 kg to about 500 g/1000 kg of complete feed, and the amount of mannans and/or manno-proteins in the animal feed is in the range of about 100 g/1000 kg to about 4000 g/1000 kg of complete feed.
